(19) United States
(12) Patent Application Publication
(10) Pub. No.: US 2007/0203206 A1
(43) Pub. Date: Aug. 30, 2007

(54) 2-Pyrrolidone Derivatives And Use Thereof As Factor Xa Inhibitors

(76) Inventors: Alan David Borthwick, Stevenage (GB); Savvas Kleanthous, Stevenage (GB); Stefan Senger, Stevenage (GB); Ian Edward David Smith, Stevenage (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/561,259

(22) PCT Filed: Jul. 17, 2004
(86) PCT No.: PCT/EP04/06591

§ 371 Date: Apr. 28, 2006

(30) Foreign Application Priority Data
Jun. 19, 2003 (GB) 0314370.8

(51) Int. Cl. A61K 31/426

(52) U.S. Cl. 514/365; 548/200

(57) ABSTRACT

A compound of formula (I) 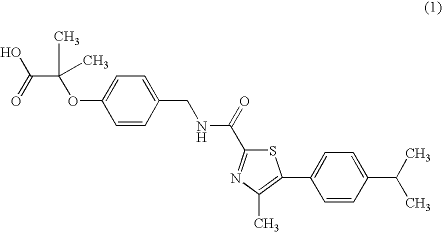 and pharmaceutically acceptable salts, solvates and hydrolysable esters thereof.

### FIELD OF THE INVENTION

The present invention relates to a novel class of chemical compounds, to processes for their preparation, to pharmaceutical compositions containing them and to their use in medicine, particularly use in the amelioration of a clinical condition for which a Factor Xa inhibitor is indicated.

### BACKGROUND OF THE INVENTION

Factor Xa is a member of the trypsin-like serine protease class of enzymes. It is a key enzyme in the coagulation cascade. A one-to-one binding of Factors Xa and Va with calcium ions and phospholipid converts prothrombin into thrombin. Thrombin plays a central role in the mechanism of blood coagulation by converting the soluble plasma protein, fibrinogen, into insoluble fibrin. The insoluble fibrin matrix is required for the stabilisation of the primary hemostatic plug. Many significant disease states are related to abnormal hemostasis. With respect to the coronary arterial vasculature, abnormal thrombus formation due to the rupture of an established atherosclerotic plaque is the major cause of acute myocardial infarction and unstable angina. Both treatment of an occlusive coronary thrombus by thrombolytic therapy and percutaneous transluminal coronary angioplasty (PTCA) are often accompanied by an acute thrombotic reclosure of the affected vessel which requires immediate resolution. With respect to the venous vasculature, a high percentage of patients undergoing major surgery in the lower extremities or the abdominal area suffer from thrombus formation in the venous vasculature which can result in reduced blood flow to the affected extremity and a predisposition to pulmonary embolism. Disseminated intravascular coagulopathy commonly occurs within both vascular systems during septic shock, certain viral infections and cancer and is characterised by the rapid consumption of coagulation factors and systemic coagulation which results in the formation of life-threatening thrombi occurring throughout the vasculature leading to widespread organ failure. Beyond its direct role in the formation of fibrin rich blood clots, thrombin has been reported to have profound bioregulatory effects on a number of cellular components within the vasculature and blood, (Shuman, M. A., Ann. NY Acad. Sci., 405: 349 (1986)).

A Factor Xa inhibitor may be useful in the treatment of acute vascular diseases such as acute coronary syndromes (for example primary and secondary prevention of myocardial infarction and unstable angina and treatment of prothrombotic sequalae associated with myocardial infarction or heart failure), thromboembolism, acute vessel closure associated with thrombolytic therapy and percutaneous transluminal coronary angioplasty, transient ischemic attacks, pulmonary embolism, deep vein thrombosis, peripheral arterial occlusion, prevention of vessel luminal narrowing (restenosis), and the prevention of thromboembolic events associated with atrial fibrillation, e.g. stroke. Factor Xa inhibitors may also be useful in preventing thrombosis and complications in patients genetically predisposed to arterial thrombosis or venous thrombosis and patients that have a disease-associated predisposition to thrombosis (e.g. type 2 diabetics). Thrombin has been reported to contribute to lung fibroblast proliferation, thus, Factor Xa inhibitors could be useful for the treatment of some pulmonary fibrotic diseases. Factor Xa inhibitors could also be useful in the treatment of tumour metastasis, by suppressing coagulation and thus preventing fibrin deposition and its concommittant facilitation of metastasis. A Factor Xa inhibitor may also have utility as an anti-inflammatory agent through its inhibition of FXa mediated activation of protease-activated receptors (PARs 1 and 2). A Factor Xa inhibitor may also have utility as an anti-atherosclerotic agent through the suppression of platelet-activation. Thrombin can induce neurite retraction and thus Factor Xa inhibitors may have potential in neurogenerative diseases such as Parkinson's and Alzheimer's disease. Factor Xa inhibitors may also have utility as anticoagulant agents in connection with the preparation, storage, fractionation or use of whole blood. They have also been reported for use in conjunction with thrombolytic agents, thus permitting the use of a lower dose of thrombolytic-agent.

### DESCRIPTION OF THE INVENTION

The present invention provides compounds of formula (I): 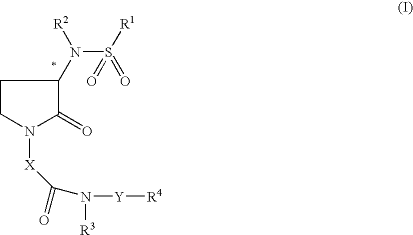
wherein:

R¹ represents a group selected from: 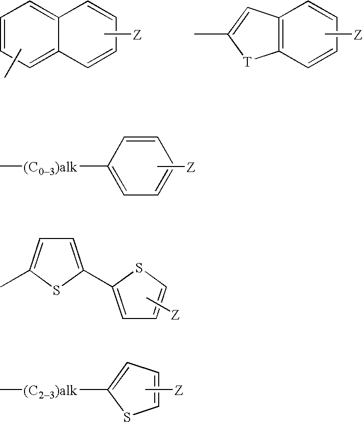
each ring of which optionally contains a further heteroatom N,

Z represents an optional substituent halogen,

alk represents alkylene or alkenylene,

T represents S, O or NH;

R² represents hydrogen, —C₁₋₆-alkyl, —C₁₋₃alkylCONR^{a}R^{b}, —C₁₋₃alkylCO₂C₁₋₄alkyl, —C₂₋₃alkylmorpholino, —CO₂C₁₋₄alkyl, or —C₁₋₃alkylCO₂H;

R^{a} and R^{b} independently represent hydrogen, —C₁₋₆alkyl, or together with the N atom to which they are bonded form a 5-, 6- or 7-membered non-aromatic heterocyclic ring optionally containing an additional heteroatom selected from O, N or S, optionally substituted by —C₁₋₄alkyl, and optionally the S heteroatom is substituted by O, i.e. represents S(O)ₙ;

n represents 0-2,

X represents phenyl or a 5- or 6-membered aromatic heterocyclic group containing at least one heteroatom selected from O, N or S, each of which is optionally substituted by 0-2 groups selected from: halogen, —C₁₋₄alkyl, —C₂₋₄alkenyl, —CN, —CF₃, —NR^{a}R^{b}, —C₀₋₄alkylOR^{e}, —C(O)R^{f} and —C(O)NR^{a}R^{b};

R^{e} represents hydrogen or —C₁₋₆-alkyl;

R^{f} represents —C₁₋₆-alkyl;

Y is absent or represents —C₁₋₃alkylene-;

R³ represents hydrogen or —C₁₋₆alkyl;

R⁴ represents —C₃₋₄alkenyl, —CH₂CH₂OH, —CH₂CO₂H, —CH₂CH₂OC₁₋₃alkyl, —CH₂CH₂SO₂C₁₋₃alkyl, —CH₂CH₂NR^{c}R^{d}, —CH₂CONR^{c}R^{d}, phenyl or a 5- or 6-membered aromatic or non-aromatic heterocyclic group containing at least one heteroatom selected from O, N or S and optionally substituted by —C₁₋₄alkyl;

R^{c} and R^{d} independently represent hydrogen, —C₁₋₆alkyl, or together with the N atom to which they are bonded form a 5-, 6- or 7-membered non-aromatic heterocyclic ring optionally containing an additional heteroatom selected from O, N or S, optionally substituted by —C₁₋₄alkyl;

and/or pharmaceutically acceptable derivative thereof.

Further aspects of the invention are:
A pharmaceutical composition comprising a compound of the invention together with a pharmaceutical carrier and/or excipient.
A compound of the invention for use in therapy.
Use of a compound of the invention for the manufacture of a medicament for the treatment of a patient suffering from a condition susceptible to amelioration by a Factor Xa inhibitor.
A method of treating a patient suffering from a condition susceptible to amelioration by a Factor Xa inhibitor comprising administering a therapeutically effective amount of a compound of the invention.

In one aspect of the invention, R¹ represents a group selected from: 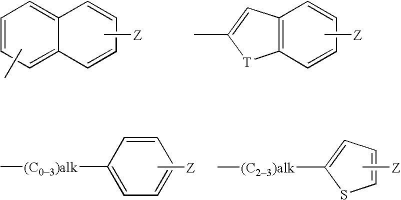
each ring of which optionally contains a further heteroatom N,

Z represents an optional substituent halogen,

alk represents alkylene or alkenylene,

T represents S, O or NH.

In another aspect, R¹ represents a group selected from: 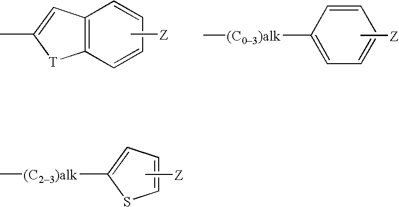
each ring of which optionally contains a further heteroatom N,

Z represents an optional substituent halogen,

alk represents alkylene or alkenylene,

T represents S, O or NH.

In another aspect of the invention, R¹ represents a group selected from: 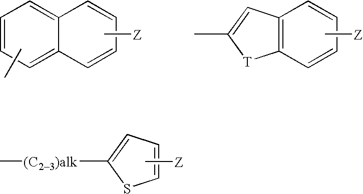
each ring of which optionally contains a further heteroatom N,

Z represents an optional substituent halogen,

alk represents alkylene or alkenylene,

T represents S, O or NH.

In another aspect, R¹ represents a group selected from:

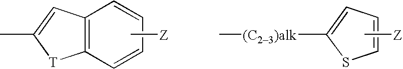

Z represents an optional substituent halogen,

alk represents alkylene or alkenylene,

T represents S, O or NH.

In another aspect of the invention, R¹ represents a group selected from: 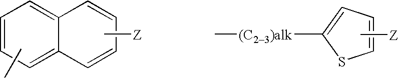
each ring of which optionally contains a further heteroatom N,

Z represents an optional substituent halogen,

alk represents alkylene or alkenylene,

T represents S, O or NH.

In another aspect, R¹ represents a group selected from:

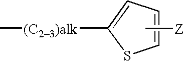

Z represents an optional substituent halogen,

alk represents alkylene or alkenylene.

In one aspect of the invention, T represents S.

In one aspect of the invention, R² represents hydrogen.

In one aspect of the invention, R^{a} and R^{b} independently represent hydrogen or —C₁₋₆alkyl.

In one aspect of the invention, X represents phenyl or a 5- or 6-membered aromatic heterocyclic group containing at least one heteroatom selected from O, N or S, each of which is optionally substituted by 0-2 groups selected from: halogen, —C₁₋₄alkyl or —NR^{a}R^{b}. In another aspect, X represents phenyl substituted by a halogen. In another aspect, X represents phenyl substituted by a fluorine.

In one aspect of the invention, Y is absent or represents C₁₋₂ alkylene.

In one aspect of the invention, R³ represents hydrogen or methyl. In another aspect, R³ represents methyl.

In one aspect of the invention, R⁴ represents —C₃₋₄alkenyl, —CH₂CH₂OH, —CH₂CO₂H, —CH₂CH₂OCH₃, —CH₂CH₂SO₂CH₃, —CH₂CH₂NR^{c}R^{d}, —CH₂CONR^{c}R^{d}, phenyl or a 5- or 6-membered aromatic heterocyclic group containing one or two heteroatoms selected from O, N or S and optionally substituted by —C₁₋₄alkyl. In another aspect, R⁴ represents —CH₂CH₂NR^{c}R^{d}.

In one aspect of the invention, R^{c} and R^{d} independently represent hydrogen, methyl or R^{c} and R^{d} together with the N atom to which they are attached form a pyrrolidine. In another aspect, R^{c} and R^{d} independently represent hydrogen or methyl.

It is to be understood that the present invention covers all combinations of the various aspects of the invention described herein above.

The compounds of formula (I) contain chiral (asymmetric) centres. The individual stereoisomers (enantiomers and diastereoisomers) and mixtures of these are within the scope of the present invention. In one aspect of the invention, the stereochemistry is (S) at the 3position on the 2-oxopyrrolidine ring (*).

As used herein, the term “alkyl” means both straight and branched chain saturated hydrocarbon groups. Examples of alkyl groups include methyl (—CH₃), ethyl (—C₂H₅), propyl (—C₃H₇) and butyl (—C₄H₉).

As used herein, the term “alkylene” means both straight and branched chain saturated hydrocarbon linker groups. Examples of alkylene groups include methylene (—CH₂—), ethylene (—CH₂CH₂—) and propylene (—CH₂CH₂CH₂—).

As used herein, the term “alkenylene” means both straight and branched chain unsaturated hydrocarbon linker groups, wherein the unsaturation is present only as double bonds. Examples of alkenylene groups includes ethenylene (—CH═CH—) and propenylene (—CH₂—CH═CH—).

As used herein, the term “heterocyclic group” means optionally substituted rings containing one or more heteroatoms selected from: nitrogen, sulphur and oxygen atoms.

The heterocycle may be aromatic or non-aromatic, i.e., may be saturated, partially or fully unsaturated. Examples of 5-membered groups include thienyl, furanyl, pyrrolidinyl thiazolyl, oxazolyl and imidazolyl. Examples of 6-membered groups include pyridyl, piperidinyl, pyrimidinyl and morpholinyl. Examples of 7-membered groups include hexamethyleneiminyl. Certain heterocyclic groups, e.g. thienyl, furanyl, thiazolyl, oxazolyl, pyridyl and pyrimidinyl are C-linked to the rest of the molecule. Other heterocyclic groups, e.g pyrrolidinyl, imidazolyl, piperidyl, morpholinyl and hexamethyleneiminyl may be C-linked or N-linked to the rest of the molecule.

As used herein, the term “halogen” means an atom selected from fluorine, chlorine, bromine and iodine.

As used herein, the term “pharmaceutically acceptable” means a compound which is suitable for pharmaceutical use.

As used herein, the term “pharmaceutically acceptable derivative”, means any pharmaceutically acceptable salt, solvate, or prodrug e.g. ester or carbamate, or salt or solvate of such a prodrug, of a compound of formula (I), which upon administration to the recipient is capable of providing (directly or indirectly) a compound of formula (I), or an active metabolite or residue thereof. Exemplary pharmaceutically acceptable derivatives are salts, solvates, esters and carbamates. More exemplary pharmaceutically acceptable derivatives are salts, solvates and esters. Even more exemplary pharmaceutically acceptable derivatives are salts and solvates.

Suitable salts according to the invention include those formed with both organic and inorganic acids and bases. Pharmaceutically acceptable acid addition salts include those formed from mineral acids such as: hydrochloric, hydrobromic, sulphuric, phosphoric, acid; and organic acids such as: citric, tartaric, lactic, pyruvic, acetic, trifluoroacetic, succinic, oxalic, formic, fumaric, maleic, oxaloacetic, methanesulphonic, ethanesulphonic, p-toluenesulphonic, benzenesulphonic and isethionic acids. Exemplary pharmaceutically acceptable salts include those formed from hydrochloric, trifluoroacetic and formic acids. Thus, in one aspect of the invention pharmaceutically acceptable salts are formic acid salts.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of formula (I) are within the scope of the invention.

Salts and solvates of compounds of formula (I) which are suitable for use in medicine are those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of formula (I) and their pharmaceutically acceptable salts and solvates.

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxyl or amine groups are bonded to any group that, when administered to a patient, cleaves to form the hydroxyl or amine groups.

Esters may be active in their own right and/or be hydrolysable under in vivo conditions in the human body. Suitable pharmaceutically acceptable in vivo hydrolysable ester groups include those which break down readily in the human body to leave the parent acid or its salt. An ester may be formed at a carboxylic acid (—COOH) group or a hydroxyl (—OH) group, by methods well known in the art involving reaction with the corresponding alcohol, acid, acid chloride, anhydride, or amide. Suitable esters are C₁₋₆alkyl esters, e.g. methyl esters, ethyl esters, and the like.

Exemplary compounds of the invention include:
4-[3-({[(1E)-2-(5-Chloro-2-thienyl)-1-propen-1-yl]sulfonyl}amino)-2-oxo-1-pyrrolidinyl]-3-fluoro-N-methyl-N-[2-(methylamino)ethyl]benzamide;
4-[3-({[(1E)-2-(5-Chloro-2-thienyl)-1-propen-1-yl]sulfonyl}amino)-2-oxo-1-pyrrolidinyl]-3-fluoro-N-(2-hydroxyethyl)-N-methylbenzamide;
4-[3-({[(1E)-2-(5-Chloro-2-thienyl)-1-propen-1-yl]sulfonyl}amino)-2-oxo-1-pyrrolidinyl]-3-fluoro-N-methyl-N-(2-pyridinylmethyl)benzamide;
4-[3-({[(1E)-2-(5-Chloro-2-thienyl)-1-propen-1-yl]sulfonyl}amino)-2-oxo-1-pyrrolidinyl]-3-fluoro-N-methyl-N-[2-(methylsulfonyl)ethyl]benzamide;
4-[3-({[(1E)-2-(5-Chloro-2-thienyl)-1-propen-1-yl]sulfonyl}amino)-2-oxo-1-pyrrolidinyl]-3-fluoro-N-methyl-N-[2-(methyloxy)ethyl]benzamide;
4-[3-({[(E)-2-(5-Chloro-2-thienyl)ethenyl]sulfonyl}amino)-2-oxo-1-pyrrolidinyl]-3-fluoro-N-methyl-N-[2-(3-pyridinyl)ethyl]benzamide;
4-[3-({[(E)-2-(5-Chloro-2-thienyl)ethenyl]sulfonyl}amino)-2-oxo-1-pyrrolidinyl]-3-fluoro-N-methyl-N-(2-phenylethyl)benzamide;
4-[3-({[(E)-2-(5-Chloro-2-thienyl)ethenyl]sulfonyl}amino)-2-oxo-1-pyrrolidinyl]-3-fluoro-N-(4-pyridinylmethyl)benzamide;
4-[3-({[(E)-2-(5-Chloro-2-thienyl)ethenyl]sulfonyl}amino)-2-oxo-1-pyrrolidinyl]-3-fluoro-N-(3-pyridinylmethyl)benzamide;
4-[3-({[(E)-2-(5-Chloro-2-thienyl)ethenyl]sulfonyl}amino)-2-oxo-1-pyrrolidinyl]-3-fluoro-N-(2-hydroxyethyl)-N-methylbenzamide;
4-[3-({[(E)-2-(5-Chloro-2-thienyl)ethenyl]sulfonyl}amino)-2-oxo-1-pyrrolidinyl]-3-fluoro-N-(phenylmethyl)benzamide;
4-[3-({[(E)-2-(5-Chloro-2-thienyl)ethenyl]sulfonyl}amino)-2-oxo-1-pyrrolidinyl]-3-fluoro-N-methyl-N-[2-(methyloxy)ethyl]benzamide;
4-[3-({[(E)-2-(5-Chloro-2-thienyl)ethenyl]sulfonyl}amino)-2-oxo-1-pyrrolidinyl]-N-[2-(dimethylamino)ethyl]-3-fluoro-N-methylbenzamide;
4-[3-({[(E)-2-(5-Chloro-2-thienyl)ethenyl]sulfonyl}amino)-2-oxo-1-pyrrolidinyl]-3-fluoro-N-methyl-N-[2-(methylsulfonyl)ethyl]benzamide;
4-[3-({[(E)-2-(5-Chloro-2-thienyl)ethenyl]sulfonyl}amino)-2-oxo-1-pyrrolidinyl]-3-fluoro-N-methyl-N-2-propen-1-ylbenzamide;
N-(2-Amino-2-oxoethyl)-4-[3-({[(E-2-(5-chloro-2-thienyl)ethenyl]sulfonyl}amino)-2-oxo-1-pyrrolidinyl]-3-fluoro-N-methylbenzamide;
4-[3-({[(E)-2-(5-Chloro-2-thienyl)ethenyl]sulfonyl}amino)-2-oxo-1-pyrrolidinyl]-3-fluoro-N-methyl-N-(4-pyridinylmethyl)benzamide;
4-[3-({[(E)-2-(5-Chloro-2-thienyl)ethenyl]sulfonyl}amino)-2-oxo-1-pyrrolidinyl]-3-fluoro-N-methyl-N-[2-(1-pyrrolidinyl)ethyl]benzamide;
4-[3({[(E)-2-(5-Chloro-2-thienyl)ethenyl]sulfonyl}amino)-2-oxo-1-pyrrolidinyl]-3-fluoro-N-[2-(1H-imidazol-4-yl)ethyl]-N-methylbenzamide;
4-[3-({[(E)-2-(5-Chloro-2-thienyl)ethenyl]sulfonyl}amino)-2-oxo-1-pyrrolidinyl]-3-fluoro-N-(3-hydroxypropyl)-N-methylbenzamide;
4-[3-({[(E)-2-(5-Chloro-2-thienyl)ethenyl]sulfonyl}amino)-2-oxo-1-pyrrolidinyl]-3-fluoro-N-methyl-N-[3-(methylamino)-3-oxopropyl]benzamide;
4-[3-({[(E)-2-(5-Chloro-2-thienyl)ethenyl]sulfonyl}amino)-2-oxo-1-pyrrolidinyl]-3-fluoro-N-methyl-N-[2-(4-methyl-1H-imidazol-5-yl)ethyl]benzamide;
N-({4-[3-({[(E)-2-(5-Chloro-2-thienyl)ethenyl]sulfonyl}amino)-2-oxo-1-pyrrolidinyl]-3-fluorophenyl}carbonyl)-N-methylglycine;
N-({4-[3-({[(E)-2-(5-Chloro-2-thienyl)ethenyl]sulfonyl}amino)-2-oxo-1-pyrrolidinyl]-3-fluorophenyl}carbonyl)glycine;
4-(3-{[(6-Chloro-1-benzothien-2-yl)sulfonyl]amino}2-oxo-1-pyrrolidinyl)-N-[2-(dimethylamino)ethyl]-3-fluoro-N-methylbenzamide;
4-(3-{[(6-Chloro-1-benzothien-2-yl)sulfonyl]amino}-2-oxo-1-pyrrolidinyl)-3-fluoro-N-methyl-N-[2-(methylamino)ethyl]benzamide;
4-(3-{[(6-Chloro-1-benzothien-2-yl)sulfonyl]amino}-2-oxo-1-pyrrolidinyl)-3-fluoro-N-methyl-N-[2-(3-pyridinyl)ethyl]benzamide;
N-(2-Aminoethyl)-4-(3-{[(6-chloro-1-benzothien-2-yl)sulfonyl]amino}-2-oxo-1-pyrrolidinyl)-3-fluoro-N-methylbenzamide;
4-[3-({[(1E)-2-(5-Chloro-2-thienyl)-1-propen-1-yl]sulfonyl}amino)-2-oxo-1-pyrrolidinyl]-N-[2-(dimethylamino)ethyl]-3-fluoro-N-methylbenzamide;
4-[3-({[(1E)-2-(5-Chloro-2-thienyl)-1-propen-1-yl]sulfonyl}amino)-2-oxo-1-pyrrolidinyl]-3-fluoro-N-methyl-N-[2-(3-pyridinyl)ethyl]benzamide;
4-[3-({[(1E)-2-(5-Chloro-2-thienyl)-1-propen-1-yl]sulfonyl}amino)-2-oxo-1-pyrrolidinyl]-3-fluoro-N-[2-(1H-imidazol-4-yl)ethyl]-N-methylbenzamide; and
4-(3-{[(6-Chloro-2-naphthalenyl)sulfonyl]amino}-2-oxo-1-pyrrolidinyl)-3-fluoro-N-methyl-N-[2-(methylamino)ethyl]benzamide.

Compounds of the invention may show advantageous properties, they may be more efficacious, may show greater selectivity, may have fewer side effects, may have a longer duration of action, may be more bioavailable by the preferred route, or may have other more desirable properties than similar known compounds.

The compounds of formula (I) are Factor Xa inhibitors and as such are useful in the treatment of clinical conditions susceptible to amelioration by administration of a Factor Xa inhibitor. Such conditions include acute vascular diseases such as acute coronary syndromes (for example primary and secondary prevention of myocardial infarction and unstable angina and treatment of prothrombotic sequalae associated with myocardial infarction or heart failure), thromboembolism, acute vessel closure associated with thrombolytic therapy and percutaneous transluminal coronary angioplasty (PTCA), transient ischemic attacks, pulmonary embolism, deep vein thrombosis, peripheral arterial occlusion, prevention of vessel luminal narrowing (restenosis), and the prevention of thromboembolic events associated with atrial fibrillation, e.g. stroke; in preventing thrombosis and complications in patients genetically predisposed to arterial thrombosis or venous thrombosis and patients that have a disease-associated predisposition to thrombosis (e.g. type 2 diabetics); the treatment of pulmonary fibrosis; the treatment of tumour metastasis; inflammation; atherosclerosis; neurogenerative disease such as Parkinson's and Alzheimer's diseases; Kasabach Merritt Syndrome; Haemolytic uremic syndrome; endothelial dysfunction; as anti-coagulants for extracorporeal blood in for example, dialysis, blood filtration, bypass, and blood product storage; and in the coating of invasive devices such as prostheses, artificial valves and catheters in reducing the risk of thrombus formation.

Accordingly, one aspect of the present invention provides a compound of formula (I) and/or a pharmaceutically acceptable derivative thereof for use in medical therapy, particularly for use in the amelioration of a clinical condition in a mammal, including a human, for which a Factor Xa inhibitor is indicated.

In another aspect, the invention provides a method for the treatment and/or prophylaxis of a mammal, including a human, suffering from a condition susceptible to amelioration by a Factor Xa inhibitor which method comprises administering to the subject an effective amount of a compound of formula (I) and/or a pharmaceutically acceptable derivative thereof.

In another aspect, the present invention provides the use of a compound of formula (I) and/or a pharmaceutically acceptable derivative thereof, for the manufacture of a medicament for the treatment and/or prophylaxis of a condition susceptible to amelioration by a Factor Xa inhibitor.

In one aspect of the invention, the condition susceptible to amelioration by a Factor Xa inhibitor is selected from treatment of acute vascular diseases such as acute coronary syndromes (for example primary and secondary prevention of myocardial infarction and unstable angina and treatment of prothrombotic sequalae associated with myocardial infarction or heart failure), thromboembolism, acute vessel closure associated with thrombolytic therapy and percutaneous transluminal coronary angioplasty, transient ischemic attacks, pulmonary embolism, deep vein thrombosis, peripheral arterial occlusion, prevention of vessel luminal narrowing (restenosis), and the prevention of thromboembolic events associated with atrial fibrillation, e.g. stroke.

In another aspect, the condition susceptible to amelioration by a Factor Xa inhibitor is selected from acute coronary syndromes (for example primary and secondary prevention of myocardial infarction and unstable angina and treatment of prothrombotic sequalae associated with myocardial infarction or heart failure), pulmonary embolism, deep vein thrombosis and the prevention of thromboembolic events associated with atrial fibrillation, e.g. stroke.

It will be appreciated that reference to treatment includes acute treatment or prophylaxis as well as the alleviation of established symptoms.

While it is possible that, for use in therapy, a compound of the present invention may be administered as the raw chemical, it is preferable to present the active ingredient as a pharmaceutical formulation.

In a further aspect, the invention provides a pharmaceutical composition comprising at least one compound of formula (I) and/or a pharmaceutically acceptable derivative thereof in association with a pharmaceutically acceptable carrier and/or excipient. The carrier and/or excipient must be “acceptable” in the sense of being compatible with the other ingredients of the formulation and not deleterious to the recipient thereof.

Accordingly, the present invention further provides a pharmaceutical formulation comprising at least one compound of formula (I) and/or a pharmaceutically acceptable derivative thereof, in association with a pharmaceutically acceptable carrier and/or excipient. The carrier and/or excipient must be “acceptable” in the sense of being compatible with the other ingredients of the formulation and not deleterious to the recipient thereof.

In another aspect, the invention provides a pharmaceutical composition comprising, as active ingredient, at least one compound of formula (I) and/or a pharmaceutically acceptable derivative thereof in association with a pharmaceutically acceptable carrier and/or excipient for use in therapy, and in particular in the treatment of human or animal subjects suffering from a condition susceptible to amelioration by a Factor Xa inhibitor.

There is further provided by the present invention a process of preparing a pharmaceutical composition, which process comprises mixing at least one compound of formula (I) or a pharmaceutically acceptable derivative thereof, together with a pharmaceutically acceptable carrier and/or excipient.

The compounds for use according to the present invention may be formulated for oral, buccal, parenteral, topical, rectal or transdermal administration or in a form suitable for administration by inhalation or insufflation (either through the mouth or the nose).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulfate). The tablets may be coated by methods well known in the art. Liquid preparations for oral administration may take the form of, for example, solutions, syrups or suspensions or they may be presented as a dry product for constitution with water or other suitable vehicles before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Preparations for oral administration may be suitably formulated to give controlled/extended release of the active compound.

For buccal administration the compositions may take the form of tablets or lozenges formulated in a conventional manner.

The compounds according to the present invention may be formulated for parenteral administration by injection, e.g. by bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use.

The compounds according to the present invention may be formulated for topical administration by insufflation and inhalation. Examples of types of preparation for topical administration include sprays and aerosols for use in an inhaler or insufflator.

Powders for external application may be formed with the aid of any suitable powder base, for example, lactose, talc or starch. Spray compositions may be formulated as aqueous solutions or suspensions or as aerosols delivered from pressurised packs, such as metered dose inhalers, with the use of a suitable propellant.

The compounds according to the present invention may also be formulated in rectal compositions such as suppositories or retention enemas, e.g. containing conventional suppository bases such as cocoa butter or other glycerides.

In addition to the formulations described previously, the compounds may also be formulated as a depot preparation. Such long acting formulations may be administered by implantation (for example subcutaneously, transcutaneously or intramuscularly) or by intramuscular injection. Thus, for example, the compounds according to the present invention may be formulated with suitable polymeric or hydrophobic materials (for example as an emulsion in an acceptable oil) or ion exchange resins or as sparingly soluble derivatives, for example, as a sparingly soluble salt.

A proposed dose of the compounds according to the present invention for administration to a human (of approximately 70 kg body weight) is 0.1 mg to 1 g, such as 1 mg to 500 mg of the active ingredient per unit dose, expressed as the weight of free base. The unit dose may be administered, for example, 1 to 4 times per day. The dose will depend on the route of administration. It will be appreciated that it may be necessary to make routine variations to the dosage depending on the age and weight of the patient as well as the severity of the condition to be treated. The dosage will also depend on the route of administration. The precise dose and route of administration will ultimately be at the discretion of the attendant physician or veterinarian.

The compounds of formula (I) may also be used in combination with other therapeutic agents. The invention thus provides, in a further aspect, a combination comprising a compound of formula (I) and/or a pharmaceutically acceptable derivative thereof together with one or more further therapeutic agent(s).

When a compound of formula (I) and/or a pharmaceutically acceptable derivative thereof is used in combination with a second therapeutic agent the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. The compounds of the present invention may be used in combination with other antithrombotic drugs (such as thrombin inhibitors, thromboxane receptor antagonists, prostacyclin mimetics, phosphodiesterase inhibitors, fibrinogen antagonists, thrombolytic drugs such as tissue plasminogen activator and streptokinase, non-steroidal anti-inflammatory drugs such as aspirin, and the like), anti-hypertensive agents (such as angiotensin-converting enzyme inhibitors, angiotensin-II receptor antagonists, ACE/NEP inhibitors, β-blockers, calcium channel blockers, PDE inhibitors, aldosterone blockers), anti-atherosclerotic/dyslipidaemic agents (such as HMG-CoA reductase inhibitors) and anti-arrhythmic agents.

The combinations referred to above may conveniently be presented for use in the form of a pharmaceutical formulation and thus pharmaceutical formulations comprising a combination as defined above together with at least one pharmaceutically acceptable carrier and/or excipient comprise a further aspect of the invention. The individual components of such combinations may be administered either sequentially or simultaneously in separate or combined pharmaceutical formulations by any convenient route.

When administration is sequential, either the Factor Xa inhibitor or the second therapeutic agent may be administered first. When administration is simultaneous the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

The compounds of formula (I) and/or pharmaceutically acceptable derivatives thereof may be prepared by the processes described hereinafter, said processes constituting a further aspect of the invention. In the following description, the groups are as defined above for compounds of formula (I) unless otherwise stated.

According to a further aspect of the present invention, there is provided a process (A) for preparing a compound of formula (I) which comprises reacting compound of formula (II) or an acid addition salt thereof with a compound of formula (III) where V is a suitable leaving group, such as a halide, e.g. chloride. When the free base of a compound of formula (II) is used, the reaction is conveniently carried out in the presence of a base, e.g. pyridine, and in a suitable solvent, e.g. acetonitrile (MeCN), suitably at 0° C. to room temperature. When the acid addition salt of a compound of formula (II) is used, the reaction is conveniently carried out in the presence of a base, e.g. N,N-diisopropylethylamine (DIPEA), and in a suitable solvent, e.g. MeCN, suitably at 0° C. to room temperature.

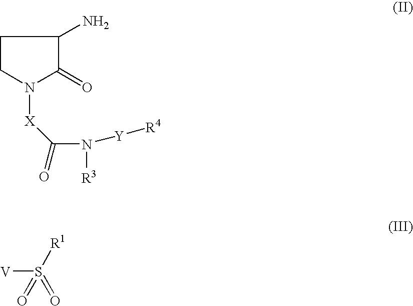

If the group X—CON(R³)(YR⁴) contains a group reactive to compounds of formula (III), such groups may be protected prior to reaction of a compound of formula (II) with a compound of formula (III) using methods well known in the art and such protecting groups removed under standard conditions to provide compounds of formula (I) after completion of the reaction of a compound of formula (II) with a compound of formula (III).

Compounds of formula (III) are known compounds or may be prepared by methods known in the literature or processes known to those skilled in the art.

Compounds of formula (II) may be prepared from compounds of formula (IV): 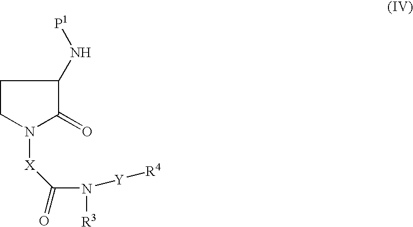
wherein P¹ is a suitable amine protecting group, e.g. Boc (t-butyloxycarbonyl), by removal of the protecting group under standard conditions. For example, when P¹ represents Boc, removal of the protecting group may be effected under acidic conditions, using for example trifluoroacetic acid (TFA) in a solvent such as dichloromethane (DCM), suitably at room temperature. If as described above, the group X—CON(R³)(YR⁴) contains a group reactive to compounds of formula (III), such groups may be protected prior to formation of a compound of formula (II).

Compounds of formula (IV) may be prepared by reaction of compounds of formula (V) with compounds of formula (VI) or an acid addition salt thereof: 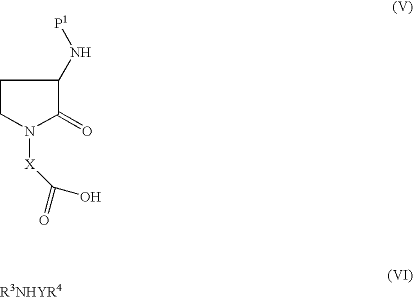
in a suitable solvent, e.g. N,N-dimethylformamide (DMF), in the presence of a base, e.g. N,N-diisopropylethylamine (DIPEA), and in the presence of a carboxylic acid activating agent, e.g. N-[1H-1,2,3-benzotriazol-1-yloxy(dimethylamino)methylidene]-N-methylmethanaminium tetrafluoroborate (TBTU), suitably at room temperature. It will be understood by persons skilled in the art, that if R³ and R⁴ contain a group(s) reactive to reagents used during the above reaction conditions, such groups may be protected prior to using, methods well known in the art and removed under standard conditions after completion of the reaction.

Compounds of formula (VI) are known compounds or may be prepared by methods known in the literature or processes known to those skilled in the art.

Compounds of formula (V) may be prepared from compounds of formula (VII): 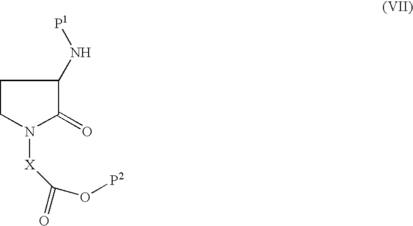
where P² is a suitable carboxylic acid protecting group e.g. benzyl, by removal of the protecting group under standard conditions. For example, when P² represents benzyl, removal of the protecting group may be effected by hydrogenolysis using a suitable catalyst, e.g. 10% palladium on carbon, in a suitable solvent such as ethanol, suitably at atmospheric pressure and room temperature.

Compounds of formula (VII) may be prepared from compounds of formula (VIII): 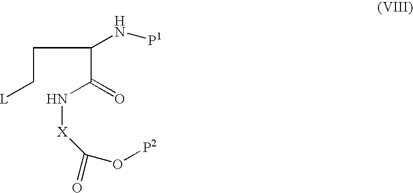
where L represents a suitable leaving group, e.g. hydroxyl, by cyclisation. For example, when L is a hydroxyl group, the ring closure may be performed by treatment with a mixture of (i) aryl or alkyl phosphine, e.g. tri-n-butylphosphine, and (ii) a suitable azodicarboxylate derivative, e.g. 1,1′-(azodicarbonyl)-dipiperidine, in a suitable solvent, e.g. tetrahydrofuran (THF), suitably at 0° C. to room temperature.

It will be appreciated by persons skilled in the art that compounds of formula (VIII) may be prepared by interconversion, utilising other compounds of formula (VIII) which are optionally protected by standard protecting groups, as precursors. For instance, compounds of formula (VIII) where L is OH, may be converted into compounds of formula (VIII) possessing alternative substituents at L, e.g. halogen, S⁺MeR W⁻ or OSO₂R, by methods well known in the art (see for example Smith, M. B. and March, J., Advanced Organic Chemistry, 5^{th} Edition 2001, John Wiley & Sons). Generally R will represent alkyl or aralkyl and W will represent sulfate or halide, especially iodide. In such cases the ring closure may be performed by treatment with a base in a suitable solvent, e.g. MeCN.

Compounds of formula (VIII), where L is a hydroxyl group, may be prepared by reacting a compound of formula (IX) with a compound of formula (X): 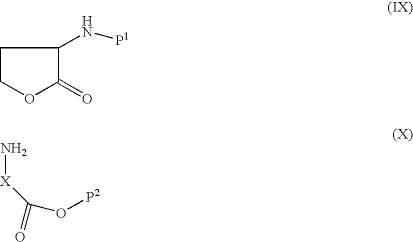
where P¹ and P² are a suitable protecting groups as described above. The reaction is conveniently carried out by addition of a suitable activating agent, e.g. trimethylaluminium, to compounds of formula (X) in a suitable solvent, e.g. DCM, under an inert atmosphere, e.g. nitrogen, suitably at room temperature followed by addition of a compounds of formula (IX) in a compatible solvent, e.g. DCM.

Compounds of formula (IX) may be prepared from compounds of formula (XI) where HA is a suitable acid, e.g. hydrochloric acid, using methods well known to those skilled in the art. See, for example, “Protective groups in organic synthesis” by T. W. Greene and P. G. M. Wuts (John Wiley & sons 1991) or “Protecting Groups” by P. J. Kocienski (Georg Thieme Verlag 1994).

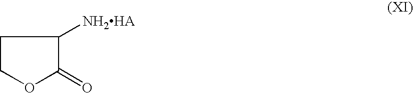

Compounds of formula (XI) are known compounds or may be prepared by methods known in the literature or processes known to those skilled in the art.

Compounds of formula (X) may be prepared by methods known in the art, e.g. from compounds of formula (XII): 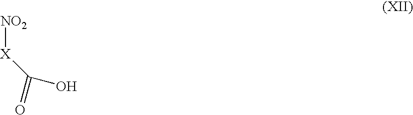
by introduction of P² under standard conditions e.g. where P² is benzyl the reaction may be performed by reaction of a compound (XII) with benzyl bromide, in a suitable solvent, e.g. DMF, in the presence of a base, e.g. sodium bicarbonate (NaHCO₃), suitably at 0° C. to room temperature, followed by reduction in the presence of a suitable reductant, such as tin(II)chloride dihydrate, in a suitable solvent, e.g. ethyl acetate, suitably under reflux.

Compounds of formula (XII) are known compounds or may be prepared by methods known in the literature or processes known to those skilled in the art.

There is provided a further process (B) for preparing compounds of formula (I) where R² is hydrogen, by reacting compounds of formula (XIII) with compounds of formula (VI): 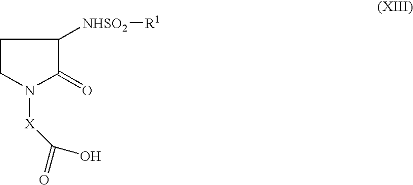
in a suitable solvent, e.g. DMF, optionally in the presence of a base, e.g. DIPEA or triethylamine, and in the presence of a carboxylic acid activating agent, e.g. TBTU. It will be understood by persons skilled in the art, that if R³ and R⁴ contain a group(s) reactive to reagents used during the above reaction conditions, such groups may be protected prior to using methods well known in the art and removed under standard conditions after completion of the reaction.

Compounds of formula (XIII) may be prepared by reaction of compounds of formula (XIV) or an acid addition salt thereof with compounds of formula (III): 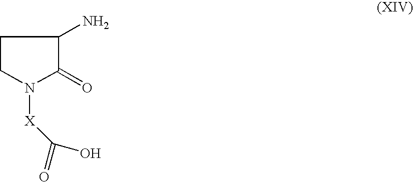
in the presence of a base, e.g. pyridine, and in a suitable solvent, e.g. MeCN, suitably at room temperature.

compound of formula (XIV) may be prepared from a compound of formula (XV): 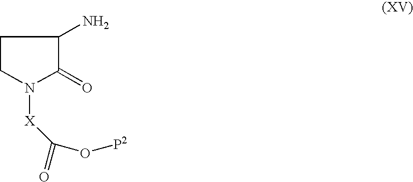
where P² is a carboxylic acid protecting group, e.g. benzyl, by removal of the protecting group under standard conditions. For example, where P² represents benzyl removal may be effected by hydrogenolysis using a suitable catalyst, e.g. 20% palladium hydroxide on carbon, in a suitable solvent such as ethanol, suitably at atomospheric pressure and room temperature, and optionally in the presence of a suitable cosolvent, e.g. acetic acid.

A compound of formula (XV) may be prepared from a compound of formula (VII) by removal of the protecting group under standard conditions. For example, where P¹ represents Boc, removal of the protecting group P¹ may be effected under standard conditions, using for example TFA in a solvent such as DCM or hydrogen chloride in dioxan, suitably at room temperature.

Alternatively, a compound of formula (XIII) may be prepared from a compound of formula (XVI): 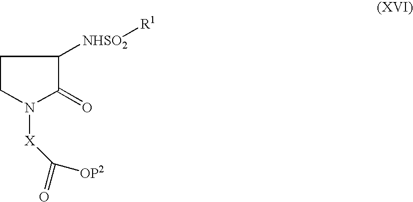
where P² represents a carboxylic acid protecting group e.g. benzyl, by removal of the protecting group under standard conditions. For example, where P² is benzyl, removal of the protecting group may be effected by treatment with an aqueous solution of hydroxide, e.g. lithium hydroxide or sodium hydroxide, in a suitable solvent, e.g. THF or methanol and suitably at room temperature to 70° C.

A compound of formula (XVI) may be prepared by reacting a compound of formula (XV) with a compound of formula (III) in a suitable solvent such as MeCN, in the presence of a base, e.g. DIPEA and optionally pyridine.

There is provided a further process (C) for preparing compounds of formula (I) where R² is other than hydrogen which comprises reacting a compound of formula (I) where R² is hydrogen with a compound of formula (XVII):
R²-T (XVII)
where R² is —C₁₋₆alkyl, —C₁₋₃alkylCONR^{a}R^{b}, —C₁₋₃alkylCO₂C₁₋₄-alkyl, —C₂₋₃alkylmorpholino or —CO₂C₁₋₄alkyl and T is a suitable leaving group such as that derived from a hydroxyl group or a halide, e.g. bromide, optionally followed by removal of the alkyl protecting group, e.g. t-Butyl, under standard conditions to form a compound wherein R² is C₁₋₃alkylCO₂H. When T is halide, the reaction is effected in a suitable organic solvent, e.g. THF or DMF, in the presence of a base, e.g. LiHMDS (lithium hexamethyldisilylamide), potassium carbonate or sodium carbonate, at a temperature range from −78° C. to +50° C., e.g. −78° C. to room temperature. When T is a hydroxyl group, the reaction is effected under Mitsunobu conditions (for examples see Hughes, David L. Progress in the Mitsunobu reaction. A review. Organic Preparations and Procedures International (1996), 28(2), 127-64.). For example, the reaction may be performed by treatment of compounds of formula (I) where R² represents H with an aryl or alkyl phosphine, e.g. triphenylphosphine, optionally bound to polymer-support, and an azodicarboxylate derivative, e.g. di-tert-butyl azodicarboxylate, in a suitable solvent, e.g. THF, followed by addition of a compound of formula (XVII) where T represents OH, optionally in a suitable solvent, e.g. THF, suitably at room temperature.

When the group X—CON(R³)(YR⁴) contains a group reactive to compounds of formula (XVII), such groups may be protected prior to the reaction using methods well known in the art and such protecting groups removed under standard conditions to provide compounds of formula (I) where R² is a substituent other than hydrogen after completion of the reaction of a compound of formula (I) where R² is hydrogen with a compound of formula (XVII).

Compounds of formula (XVII) are known compounds or may be prepared by methods known in the literature or processes known to those skilled in the art.

Furthermore, it will appreciated that the substituent R², other than hydrogen, may be introduced at various intermediate stages by methods well known to those skilled in the art.

There is provided a further process (D) for preparing compounds of formula (VII) from a compound of formula (XVIII): 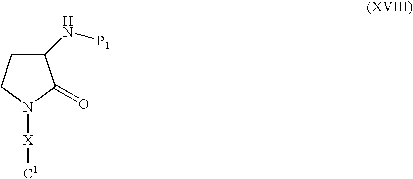
where C¹ is a suitable coupling group, such as halide, e.g. iodide, by carbonylation with carbon monoxide in the presence of a metal catalyst, e.g. palladium acetate or palladium chloride, a base, e.g. triethylamine, and a suitable alcoholic solvent, P₂OH, e.g. benzyl alcohol, optionally in the presence of a ligand, e.g. triphenylphosphine, suitably at elevated temperature, such as 40 to 200° C., e.g. at 80 to 120° C.

Compounds of formula (XVIII) may be prepared from compounds of formula (XIX): 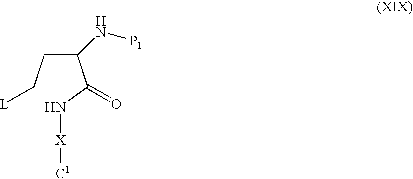
by cyclisation where L represents a leaving group, e.g. hydroxyl. For example, when L is a hydroxyl group, the ring closure may be performed by treatment with a mixture of (i) aryl or alkyl phosphine, e.g. tri-n-butylphosphine, and (ii) a suitable azodicarboxylate derivative, e.g. 1,1′-(azodicarbonyl)-dipiperidine, in a suitable solvent, e.g. THF, suitably at 0° C. to room temperature.

It will be appreciated by persons skilled in the art that compounds of formula (XIX) may be prepared by interconversion, utilising other compounds of formula (XIX) which are optionally protected by standard protecting groups, as precursors. For instance, compounds of formula (XIX) where L is OH, may be converted into compounds of formula (XIX) possessing alternative substituents at L, e.g. halogen, S⁺MeR W⁻ or OSO₂R, by methods well known in the art (see for example Smith, M. B. and March, J., Advanced Organic Chemistry, 5^{th} Edition 2001, John Wiley & Sons). Generally R will represent alkyl or aralkyl and W will represent sulfate or halide, especially iodide. In such cases the ring closure may be performed by treatment with a base in a suitable solvent, e.g. MeCN.

Compounds of formula (XIX) where L=OH, may be prepared by reacting a compound If formula (XX): 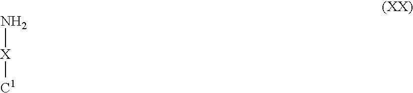
with a compound of formula (IX) wherein P¹ is a suitable protecting group as described above. The reaction is conveniently carried out in the presence of a suitable activating agent, e.g. trimethylaluminium, to compounds of formula (XX) in a suitable solvent, e.g. DCM, under an inert atmosphere, e.g. nitrogen, suitably at 0° C. to room temperature followed by addition of a compound of formula (IX) in a compatible solvent, e.g. DCM.

Compounds of formula (XX) are known compounds or may be prepared by methods known in the literature or processes known to those skilled in the art.

It will be appreciated by those skilled in the art that compounds of formula (I) or a solvate thereof may be synthesized from appropriate intermediates via solid phase chemistry processes.

Those skilled in the art will appreciate that in the preparation of the compound of formula (I) or a solvate thereof it may be necessary and/or desirable to protect one or more sensitive groups in the molecule or the appropriate intermediate to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl or aralkyl type protecting groups (e.g. benzyl, trityl, chlorotrityl). Examples of suitable hydroxyl protecting groups may include for example alkyl silyl groups, such as trimethylsilyl or tert-butyldimethylsilyl; alkyl ethers such as tetrahydropyranyl or tert-butyl; or esters such as acetate. Examples of carboxylic acid protecting groups may include for example aralkyl groups, e.g. benzyl, or alkyl groups, e.g. t-butyl.

Various intermediate compounds used in the above-mentioned process, including but not limited to certain compounds of formulae (II) and (IV) constitute a further aspect of the present invention.

The present invention will now be further illustrated by the accompanying examples which should not be construed as limiting the scope of the invention in any way.

All publications, including but not limited to patents and patent applications, cited in this specification are herein incorporated by reference as if each individual publication were specifically and individually indicated to be incorporated by reference herein as though fully set forth.

### EXAMPLES

### Abbreviations

THF Tetrahydrofuran
DCM Dichloromethane
DMF N,N-dimethylformamide
DMSO Dimethylsulphoxide
EtOAc Ethyl acetate
Ether Diethyl ether
MeOH Methanol
MeCN Acetonitrile
DIPEA N,N-diisopropylethylamine
TBTU N-[1H-1,2,3-benzotriazol-1-yloxy(dimethylamino)methylidene]-N-methylmethanaminium tetrafluoroborate
TEA Triethylamine
TFA Trifluoroacetic acid
CBZ carbobenzyloxy
br broad
m multiplet
q quartet
s singlet
t triplet
d doublet
dd double doublet
   Intermediate 1

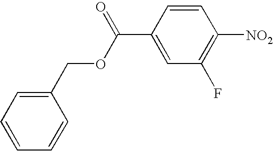

### Phenylmethyl 3-fluoro-4-nitrobenzoate

A suspension of 3-fluoro-4-nitrobenzoic acid (33.0 g) in anhydrous DMF (300 ml) was cooled to 0° C., treated with sodium bicarbonate (13.5 g) and stirred for 30 min. The mixture was treated dropwise with benzyl bromide (19.0 ml) stirred for a further 30 min then allowed to warm to room temperature while stirring overnight (18 h). The resulting orange solution was evaporated under reduced pressure and the residue partitioned between EtOAc (1200 ml) and saturated aqueous sodium carbonate (1600 ml). The organic layer was split into 2×600 ml portions and each was washed with water (2×500 ml), brine (200 ml), dried over anhydrous magnesium sulfate then filtered. The filtrates were combined and evaporated under reduced pressure to give the title compound (38.2 g) as an orange oil which crystallised on standing overnight.

¹H NMR in CDCl₃; δ 8.11 (dd, 1H), 7.99 (m, 2H), 7.48-7.37 (5H), 5.41 (s, 2H). H.p.l.c. Rt 3.45 min.
Intermediate 2

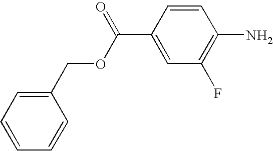

### Phenylmethyl 4-amino-3-fluorobenzoate

A solution of Intermediate 1 (38.2 g) in EtOAc (1500 ml) was treated portionwise with tin chloride dihydrate (156.5 g) over 10 min. The mixture was heated to reflux for 2.5 h then cooled to room temperature, diluted with EtOAc (1000 ml) and treated cautiously with water (1500 ml) and saturated aqueous sodium bicarbonate (1500 ml). The resulting thick suspension was stirred for 10 min then filtered through a pad of celite filteraid. Reaction vessel and filter pad were then washed with EtOAc (500 ml) and water (200 ml). The combined filtrates were partitioned, the aqueous layer extracted with EtOAc (2×1000 ml) and the combined organic layers dried over anhydrous sodium sulfate, filtered and evaporated to give the title compound (33.7 g) as an orange oil which crystallised on standing overnight.

Mass spectrum: Found: MH⁺ 246. H.p.l.c. Rt 3.25 min.
Intermediate 3

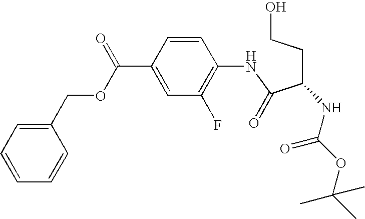

### Phenylmethyl 4-[(N-{[(1,1-dimethylethyl)oxy]carbonyl}-L-homoseryl)amino]-3-fluorobenzoate

A solution of Intermediate 2 (5.53 g) in anhydrous DCM (40 ml) at room temperature under nitrogen was treated dropwise over 35 min with trimethyl aluminium (2 molar in heptane, 13.5 ml). Slow addition rate and a water bath were used to prevent reaction temperature rising above 30° C. during addition. The mixture was stirred for 30 min then treated with a solution of 1,1-dimethylethyl [(3S)-2-oxotetrahydro-3-furanyl]carbamate (3.87 g) in anhydrous DCM (40 ml), and the final mixture stirred for 20 h before quenching by cautious addition of 5% citric acid solution (100 ml). A fine pricipitate formed, this was filtered through a pad of celite filteraid, washed with water (50 ml) and DCM (100 ml). The combined filtrates were partitioned, the aqueous layer extracted with DCM (50 ml), and the combined organic layers were washed with saturated sodium potassium tartrate solution (Rochelle's Salt)(50 ml), brine (50 ml) then dried over anhydrous magnesium sulfate, filtered and evaporated under reduced pressure to give a crude yellow oil (7.45 g). Purification by Quad Biotage™ chromatography (2×90 g Si) eluting with 1% MeOH in DCM gave the title compound (2.11 g) as a pale yellow foam.

Mass spectrum: Found: MH⁺ 447. H.p.l.c. Rt 3.27 min.
Intermediate 4

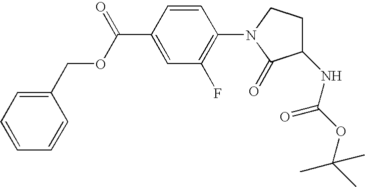

### Phenylmethyl 4-[3-{[(1,1-dimethylethyl)oxy]carbonyl}amino)-2-oxo-1-pyrrolidinyl]-3-fluorobenzoate

A solution of 1,1′(azodicarbonyl)dipiperidine (1.42 g) in anhydrous THF (15 ml) under nitrogen was cooled to 0° C. and treated with tri-n-butylphosphine (1.40 ml), giving a deep yellow solution which was stirred for 20 min. The now clear solution was treated with a solution of Intermediate 3 (2.10 g) in anhydrous THF (15 ml), giving a viscous mixture. Stirring was continued for 30 min below 5° C. and then for 20 h at room temperature. The mixture was quenched by cautious addition of 5% aqueous citric acid (20 ml) then diluted with brine (30 ml) and extracted with EtOAc (2×100 ml). The combined organic extracts were dried over anhydrous magnesium sulfate, filtered and evaporated under reduced pressure to give a white solid (5.2 g). Purification by Biotage™ chromatography (90 g, Si) eluting with cyclohexane:EtOAc (3:1) gave the title compound (2.00 g) as a white solid.

Mass spectrum: Found: MH⁺ 429. H.p.l.c. Rt 3.38 min.

Alternative Process:

Carbon monoxide was bubbled slowly to a mixture of Intermediate 21 (0.2 g) and triethylamine (0.23 ml) in benzyl alcohol (2.5 ml) at 80° C. Palladium acetate (0.03 g) was added. The reaction mixture was then warmed to 110-120° C. and stirred under carbon monoxide atmosphere at this temperature for 7 h. The cooled reaction mixture was diluted with ethyl acetate (20 ml), filtered and evaporated in vacuo. The resultant residue was partitioned between DCM (100 ml) and saturated aqueous sodium carbonate (60 ml). The organic phase was separated, washed with water (60 ml), brine (60 ml), dried over magnesium sulfate and concentrated in vacuo. Purification of the resultant residue by reverse phase SPE (2×10 g, eluting with acetonitrile:water 3:7 to 100:0) gave the title compound (0.036 g) as a white solid.

¹H-NMR δ (CDCl₃): 1.47 (s, 9H), 2.01-2.18 (m, 1H), 2.70-2.84 (bs, 1H), 3.76 (t, 1H), 3.87-3.98 (m, 1H), 4.29-4.43 (bs, 1H), 5.18 (bs, 1H), 5.36 (s, 2H), 7.33-7.47 (m, 5H), 7.61-7.65 (m, 1H), 7.82-7.91 (m, 2H).
Intermediate 5

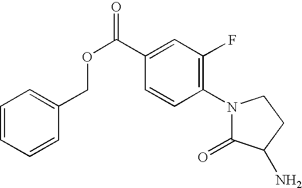

### Phenylmethyl 4-[3-amino-2-oxo-1-pyrrolidinyl]-3-fluorobenzoate

A solution of Intermediate 4 (1.5 g) in DCM (5 ml) was treated cautiously with TFA (5 ml) and the mixture stirred at ambient temperature for 2 h. The mixture was then evaporated to dryness and the residue partitioned between EtOAc (100 ml) and saturated aqueous sodium bicarbonate (100 ml). The aqueous phase was re-extracted with a further EtOAc (50 ml) and the combined organic fraction was dried (over magnesium sulfate), filtered and evaporated to give the title compound (0.83 g) as a pale brown oil.

Mass spectrum: Found: MH⁺ 329. H.p.l.c. Rt 2.28 min.
Intermediate 6

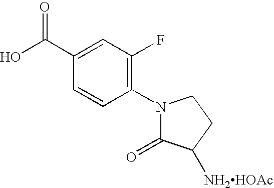

### 4-[3-Amino-2-oxo-1-pyrrolidinyl]-3-fluorobenzoic acid acetate

A solution of Intermediate 5 (0.80 g) in ethanol (40 ml) was treated with acetic acid (1 ml) and then added cautiously to 20% palladium hydroxide on carbon (0.05 g) in a flask which had been pre-purged with nitrogen. The mixture was then stirred in an atmosphere of hydrogen for 18 h after which time the mixture was purged with nitrogen, cautiously filtered and the filtrate evaporated to-provide the title compound (0.52 g) as a colourless gum.

¹H NMR (DMSO): δ13.0-13.6 (1H, broad), 8.68 (2H, s), 7.84 (1H, m), 7.68 (1H, m), 7.18 (1H, m), 4.25 (1H, m), 3.87 (1H, m), 3.44 (1H, m), 2.55 (1H, m), 2.15 (1H, m).
Intermediate 7

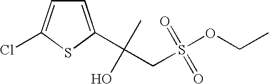

### Ethyl 2-(5-chlorothien-2-yl)-2-hydroxypropane-1-sulfonate

A solution of ethyl methanesulphonate (4.97 g) in THF (20 ml) was added dropwise to a solution of lithium hexamethyldisilylamide (42.0 ml of 1M solution in THF plus 20 ml of THF) at −78° C. under nitrogen, and the solution was stirred for 30 min. A solution of 2-acetyl-5-chlorothiophene (6.75 g) in THF (70 ml) was added to this over 15 min and the temperature maintained at −78° C. for 90 min. The reaction was quenched with saturated aqueous ammonium chloride and the mixture extracted with EtOAc. The combined organic fractions were washed with brine; dried (over magnesium sulfate) and concentrated under reduced pressure to afford a crude oil that was purified by Biotage™ chromatography (silica, eluting with ether-cyclohexane 1:3) to give the title compound (10.9 g) as a colourless oil.

¹H NMR (CDCl₃): δ6.79 (1H, d), 6.73 (1H, d), 4.26 (2H, m), 4.14 (1H, s), 3.32 (1H, d), 3.52 (1H, d), 1.8 (3H, s), 1.36 (3H, t).
Intermediate 8

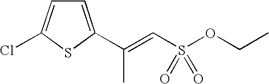

### Ethyl (1E)-2-(5-chlorothien-2-yl)prop-1-ene-1-sulfonate

A solution of Intermediate 7 (10.9 g) in DCM (300 ml) was cooled to 0° C. under nitrogen, to which was added methanesulphonic acid (15.0 ml) in a dropwise fashion. After stirring for 90 min, saturated aqueous sodium bicarbonate was added, followed by water and brine. The layers were separated and the aqueous layer back extracted with DCM; the organic fractions were combined, washed with brine and dried (over magnesium sulfate) and concentrated under reduced pressure. The crude mixture was purified using Biotage™ chromatography (silica, eluting chloroform and 15% tert-butylmethyl ether in cyclohexane) to give the title compound (2.9 g) as a white crystalline solid.

¹H NMR (CDCl₃): δ7.16 (1H, d), 6.92 (1H, d), 6.47 (1H, d), 4.26 (2H, q), 2.50 (3H, d), 1.42 (3H, t).
Intermediate 9

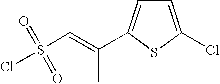

### (1E)-2-(5-Chlorothien-2-yl)prop-1-ene-1-sulfonyl chloride

Tetrabutylammonium iodide (4.03 g) was added to a solution of Intermediate 8 (2.9 g) in acetone (180 ml) under nitrogen and the solution heated under reflux for 17 h. The solution was cooled and concentrated under reduced pressure to produce a yellow-brown solid. This was stirred in phosphorus oxychloride (30 ml) at room temperature for 3.5 h, after which the volatiles were removed under reduced pressure and the residue co-evaporated twice with toluene. The residue was purified using Biotage™ chromatography (silica, eluting with, cyclohexane and cyclohexane:ether (1:1) to give the title compound (2.1 g) as a yellow crystalline solid.

¹H NMR (CDCl₃): δ7.31 (1H, d), 6.99 (1H, d), 6.96 (1H, q), 2.64 (3H, d).
Intermediate 10

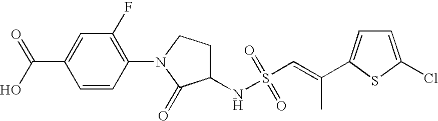

### 4-[3-({[(1E)-2-(5-Chloro-2-thienyl)-1-propen-1-yl]sulfonyl}amino)-2-oxo-1-pyrrolidinyl]-3-fluorobenzoic acid

An ice-cooled solution of Intermediate 6 (0.20 g) in MeCN (10 ml) was treated with pyridine (0.20 ml) followed by Intermediate 9 (0.172 g) and the mixture allowed to stir for 18 h. The mixture was evaporated and the residue partitioned between EtOAc (50 ml) and 1N aqueous hydrochloric acid (50 ml). The organic phase was separated, dried over magnesium sulfate, filtered and evaporated to give the title compound (0.252 g) as a brown gum.

Mass Spectrum: Found: MH⁺ 459. H.p.l.c. Rt 3.25 min.
Intermediate 11

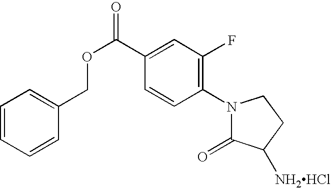

### Phenylmethyl 4-[3-amino-2-oxo-1-pyrrolidinyl]-3-fluorobenzoate hydrochloride

A solution of Intermediate 4 (0.3 g) in 1,4-dioxan (5 ml) was treated with a 4M solution of hydrogen chloride in 1,4-dioxan (3 ml). After 2 hours the mixture was evaporated to dryness to give the title compound (0.252 g) as a white solid.

Mass spectrum: Found: MH⁺ 329. H.p.l.c. Rt 2.29 min.
Intermediate 12

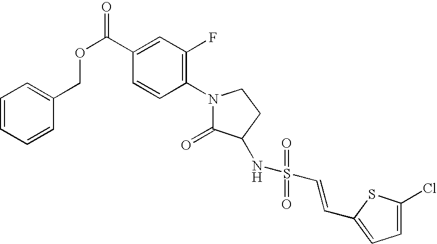

### Phenylmethyl 4-[3-({[(E)-2-(5-chloro-2-thienyl)ethenyl]sulfonyl}amino)-2-oxo-1-pyrrolidinyl]-3-fluorobenzoate

An ice-cooled suspension of Intermediate 11 (0.23 g) in anhydrous MeCN (10 ml) was treated with DIPEA (0.124 ml), pyridine (0.08 ml) and a solution of (E)-2-(5-chloro-2-thienyl)ethenesulfonyl chloride (0.18 g) in anhydrous MeCN (2 ml). The mixture was stirred for 18 h then evaporated to dryness. The residue was partitioned between EtOAc (40 ml) and saturated aqueous sodium bicarbonate (30 ml). The organic phase was dried over magnesium sulfate, filtered and evaporated to give the title compound (0.32 g) as a pale yellow solid.

Mass spectrum: Found: MH⁺ 535. H.p.l.c. Rt 3.64 min.
Intermediate 13

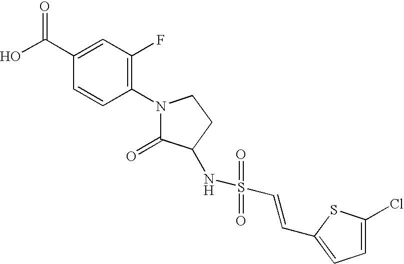

### 4-[3-({[(E)-2-(5-Chloro-2-thienyl)ethenyl]sulfonyl}amino)2-oxo-1-pyrrolidinyl]-1-3-fluorobenzoic acid

A solution of Intermediate 12 (0.05 g) in THF (5 ml) was treated with a solution of lithium hydroxide (0.006 g) in water (2.5 ml). The mixture was stirred at ambient temperature for 2.5 h then evaporated to dryness. The residue was partitioned between EtOAc (30 ml) and 2M aqueous hydrochloric acid (30 ml). The organic phase was dried over magnesium sulfate, filtered and evaporated. The residue was triturated in a mixture of ether and cyclohexane and the solid product filtered off and dried to yield the title compound (0.041 g) as a white solid.

Mass spectrum: Found: MH⁺ 445. H.p.l.c. Rt 3.14 min.
Intermediate 14

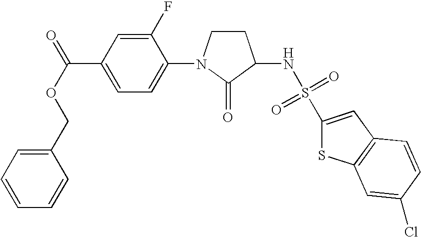

### Phenylmethyl 4-(3-{[(6-chloro-1-benzothien-2-yl)sulfonyl]amino}-2-oxo-1-pyrrolidinyl)-3-fluorobenzoate

A solution of Intermediate 5 in dry MeCN (12 ml) was cooled to 0° C. and treated with DIPEA (0.52 ml), and allowed to stir for 10 min. 6-Chloro-1-benzothiophene-2-sulfonyl chloride (0.366 g) was added in 3 portions, allowing the mixture to warm up to room temperature and stir for 18 h. The reaction was concentrated under reduced pressure, partitioning the residue between EtOAc and 1N aqueous hydrochloric acid. The separated organic layer was dried (over magnesium sulfate), concentrated under reduced pressure and the residue was purified using silica phase SPE (10 g/60 cc) eluting with cyclohexane:EtOAc gradient (10:1-2:1) to give the title compound (0.432 g) as an off white foamy solid.

Mass spectrum: Found: MH⁻ 557.
Intermediate 15

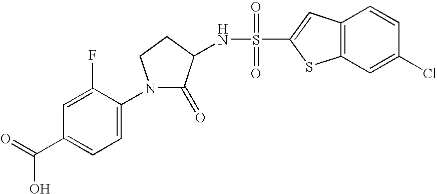

### 4-(3-{[(6-Chloro-1-benzothien-2-yl)sulfonyl]amino}-2-oxo-1-pyrrolidinyl)-3-fluorobenzoic acid

Intermediate 14 (0.43 g) in MeOH (15 ml) was treated with 1N aqueous sodium hydroxide, (1.54 ml). The mixture was heated to 60° C. for 2 h. The cooled reaction mixture was concentrated under reduced pressure, partitioning the residue between EtOAc and 1N aqueous hydrochloric acid. The separated organic layer was dried (over magnesium sulfate), concentrated under reduced pressure and the residue was purified using aminopropyl SPE (10 g/60 cc) eluting the product with 10% ammonia/MeOH to give the title compound (0.232 g) as an off white powder.

Mass spectrum: Found: MH⁺ 469.
Intermediate 16

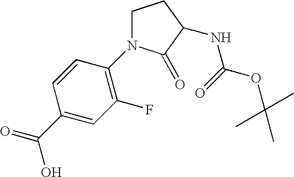

### 4-[3-({[(1,1-Dimethylethyl)oxy]carbonyl}amino)-2-oxo-1-pyrrolidinyl]-3-fluorobenzoic acid

A solution of Intermediate 4 (0.5 g) in ethanol (15 ml) was added cautiously to palladium/carbon (Degussa type E101NE/W) (0.05 g) in a flask which had been pre-purged with nitrogen. The mixture was then stirred in an atmosphere of hydrogen for 6 h after which time the mixture was purged with nitrogen and cautiously filtered through filter agent (Celite 545) washing the filter pad with ethanol. The filtrate was concentrated under reduced pressure to give the title compound (0.396 g) as a grey solid.

Mass spectrum: Found: MH⁺ 339.
Intermediate 17

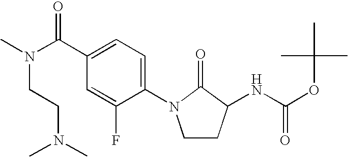

### 1,1-Dimethylethyl[1-(4-{[[2-(dimethylamino)ethyl](methyl)amino]carbonyl}-2-fluorophenyl)-2-oxo-3-pyrrolidinyl]carbamate

A solution of Intermediate 16 (0.075 g) in dry DMF (2 ml) was treated with TBTU (0.071 g) N,N,N′-trimethyl-1,2-ethanediamine (0.039 ml) and DIPEA (0.092 ml). The reaction was allowed to stir at ambient temperature for 18 h before concentrating under reduced pressure and partitioning the residue between DCM and aqueous saturated sodium bicarbonate solution. The separated organic layer was passed through a hydrophobic frit, concentrated under reduced pressure and the residue was purified by means of silica phase SPE (2 g/12 cc) eluting with DCM, ether, EtOAc & MeOH. Fractions containing product were concentrated under reduced pressure and the residue was partitioned between ether and water. The separated aqueous layer was concentrated under reduced pressure to give the title compound (0.092 g) as an orange gum.

Mass spectrum: Found: MH⁺ 423.

Using similar chemistry and Intermediate 16 the following compounds were prepared:
Intermediate 18

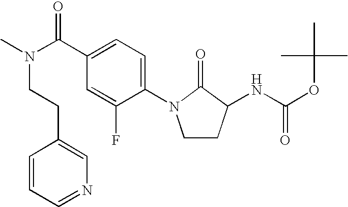

### 1,1-Dimethylethyl{1-[-2-fluoro-4-({methyl[2-(3-pyridinyl)ethyl]amino}carbonyl)phenyl]-2-oxo-3-pyrrolidinyl}carbamate

Mass spectrum: Found: MH⁺ 457.
Intermediate 19

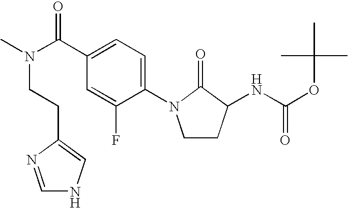

### 1,1-Dimethylethyl[1-(2-fluoro-4-{[[2-(1H-imidazol-4-yl)ethyl](methyl)amino]carbonyl}phenyl)-2-oxo-3-pyrrolidinyl]carbamate

Mass spectrum: Found: MH⁺ 446.
Intermediate 20

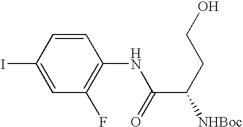

### 1,1-Dimethylethyl((1S)-1-{[(2-fluoro-4-iodophenyl)amino]carbonyl}-3-hydroxypropyl)carbamate

A solution of 2-fluoro-4-iodoaniline (7.11 g) in anhydrous DCM (40 ml) under N₂ at 0° C. was treated dropwise with trimethylaluminium (2N in heptane; 15 ml). The mixture was allowed to stir for 30 min before a solution of tert-butyl (3S)-2-oxotetrahydrofuran-3-ylcarbamate (5.03 g), in anhydrous DCM (35 ml), was added dropwise. The reaction was allowed to warm up to ambient temperature and stirred for 18 h, before quenching, with 10% aqueous citric acid acid (10 ml) Saturated aqueous potassium sodium tartrate (100 ml) was then added with stirring followed by separation of the organic and aqueous layers. The organic layer was dried (over magnesium sulfate) and concentrated under reduced pressure. The residue was purified using Biotage™ chromatography (silica, eluting with cyclohexane:ethyl acetate 3:2) to afford an off-white solid which was an inseparable mixture (c. 1:2) of the starting material and the title compound (5.55 g).

Mass spectrum: Found: MH⁺ 439.
Intermediate 21

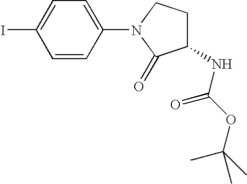

### 1,1-Dimethylethyl[(3S)-1-(2-fluoro-4-iodophenyl)-2-oxo-3-pyrrolidinyl]carbamate

To a solution of crude Intermediate 20 (5.55 g) and tri-n-butylphosphine (3.49 ml) in anhydrous THF (100 ml) under N₂ at 0° C. was added solid 1,1′-(azodicarbonyl)-dipiperidine (3.53 g). The solution was allowed to warm to ambient temperature and stirred for 18 h. The mixture was then diluted with cyclohexane (100 ml) and the precipitate filtered off. The filtrate was then concentrated under reduced pressure and the residue purified using Biotage™ chromatography (silica, eluting with cyclohexane:ethyl acetate 2:1) to give the title compound (2.93 g) as a white solid.

Mass spectrum: Found: MH⁺ 421.
Intermediate 22

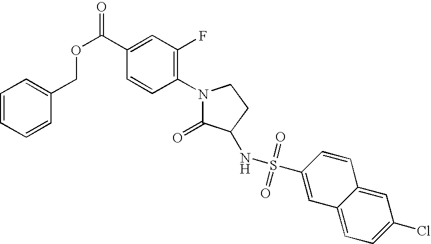

### Phenylmethyl 4-(3-{[(6-chloro-2-naphthalenyl)sulfonyl]amino}-2-oxo-1-pyrrolidinyl)-3-fluorobenzoate

Prepared according to the procedure for Intermediate 12, from Intermediate 11 and 6-chloro-2-naphthylsulfonyl chloride.

Mass spectrum: Found: MH⁺ 553. H.p.l.c. Rt 3.93 min.
Intermediate 23

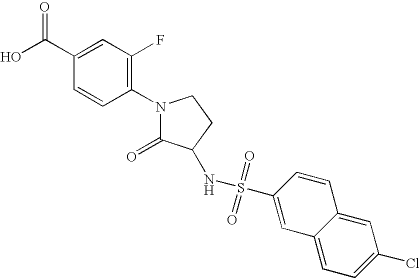

### 4-(3-{[(6-Chloro-2-naphthalenyl)sulfonyl]amino}-2-oxo-1-pyrrolidinyl)-3-fluorobenzoic acid

Prepared according to the procedure for Intermediate 13, from Intermediate 22.

Mass spectrum: Found: MH⁺ 463. H.p.l.c. Rt 3.34 min.

### Example 1

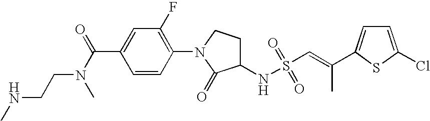

### 4-[3-({[(1E)-2-(5-Chloro-2-thienyl)-1-propen-1-yl]sulfonyl}amino)-2-oxo-1-pyrrolidinyl]-3-fluoro-N-methyl-N-[2-(methylamino)ethyl]benzamide

A solution of crude Intermediate 10 (0.040 g) in DMF (1 ml) was treated with a solution of BTU (0.031 mg) in DMF (0.5 ml) followed by a solution of N,N′-dimethyl-1,2-ethanediamine (0.035 g) in DMF (0.5 ml). The mixture was stirred overnight then evaporated to dryness. The residue was taken up in DMSO (1 ml), filtered and the desired product purified from the filtrate using mass-directed high performance liquid chromatography to provide the title compound (0.0026 g) as a white solid.

Mass spectrum: Found: MH⁺ 529. H.p.l.c. Rt 2.43 min.

Using similar chemistry and Intermediate 10, the following compounds were prepared:

### Example 2

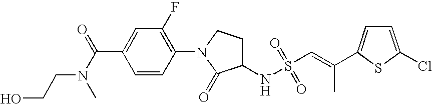

### 4-[3-({[(1E)-2-(5-Chloro-2-thienyl)-1-propen-1-yl]sulfonyl}amino)-2-oxo-1-pyrrolidinyl]-3-fluoro-N-(2-hydroxyethyl)-N-methylbenzamide

Mass spectrum: Found: M+H₂0⁺ 533. H.p.l.c. Rt 2.82 min.

### Example 3

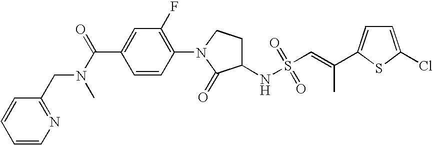

### 4-[3-({[(1E)-2-(5-Chloro-2-thienyl)-1-propen-1-yl]sulfonyl}amino)-2-oxo-1-pyrrolidinyl]-3-fluoro-N-methyl-N-(2-pyridinylmethyl)benzamide

Mass spectrum: Found: MH⁺ 563. H.p.l.c. Rt 3.02 min.

### Example 4

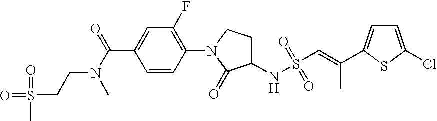

### 4-[3-({[(1E)-2-(5-Chloro-2-thienyl)-1-propen-1-yl]sulfonyl}amino)-2-oxo-1-pyrrolidinyl]-3-fluoro-N-methyl-N-[2-(methylsulfonyl)ethyl]benzamide

Mass spectrum: Found: M+H₂0⁺ 595. H.p.l.c. Rt 2.92 min.

### Example 5

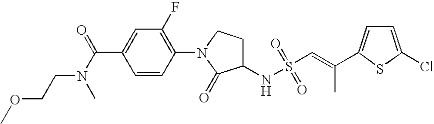

### 4-[3-({[(1E)-2-(5-Chloro-2-thienyl)-1-propen-1-yl]sulfonyl}amino)-2-oxo-1-pyrrolidinyl]-3-fluoro-N-methyl-N-[2-(methyloxy)ethyl]benzamide

Mass spectrum: Found: MH⁺ 530. H.p.l.c. Rt 3.02 min.

### Example 6

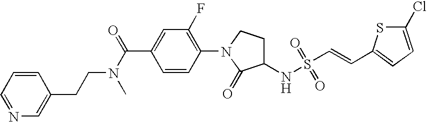

### 4-[3-({[(E)-2-(5-Chloro-2-thienyl)ethenyl]sulfonyl}amino)-2-oxo-1-pyrrolidinyl]-3-fluoro-N-methyl-N-[2-(3-pyridinyl)ethyl]benzamide

A solution of Intermediate 13 (0.025 g) in DMF (1 ml) was treated with TEA (0.025 ml), a solution of TBTU (0.02 mg) in DMF (0.5 ml) and finally with a solution of N-methyl-2-(3-pyridinyl)ethanamine (0.0102 g) in DMF (0.5 ml). The mixture was stirred at ambient for 18 h and then evaporated to dryness. The mixture was taken up in DMSO (0.5 ml), filtered and the desired product purified from the filtrate by means of mass-directed high performance liquid chromatography to give the title compound (0.0126 g) as a white solid.

Mass spectrum: Found: MH⁺ 563. H.p.l.c. Rt 2.64 min.

Using similar chemistry and Intermediate 13 the following compounds were prepared:

### Example 7

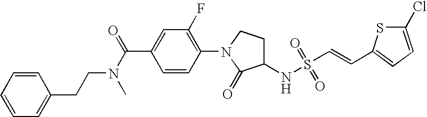

### 4-[3-({[(E)-2-(5-Chloro-2-thienyl)ethenyl]sulfonyl}amino)-2-oxo-1-pyrrolidinyl]-3-fluoro-N-methyl-N-(2-phenylethyl)benzamide

Mass spectrum: Found: MH⁺ 562. H.p.l.c. Rt 3.33 min.

### Example 8

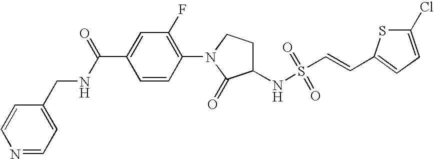

### 4-[3-({[(E)-2-(5-Chloro-2-thienyl)ethenyl]sulfonyl}amino)-2-oxo-1-pyrrolidinyl]-3-fluoro-N-(4-pyridinylmethyl)benzamide

Mass spectrum: Found: MH⁺ 535. H.p.l.c. Rt 2.57 min.

### Example 9

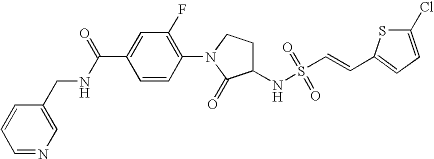

### 4-[3-({[(E)-2-(5-Chloro-2-thienyl)ethenyl]sulfonyl}amino)-2-oxo-1-pyrrolidinyl]-3-fluoro-N-(3-pyridinylmethyl)benzamide

Mass spectrum: Found: MH⁺ 535. H.p.l.c. Rt 2.69 min.

### Example 10

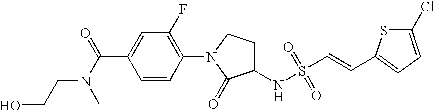

### 4-[3-({[(E)-2-(5-Chloro-2-thienyl)ethenyl]sulfonyl}amino)-2-oxo-1-pyrrolidinyl]-3-fluoro-N-(2-hydroxyethyl)-N-methylbenzamide

Mass spectrum: Found: MH⁺ 502. H.p.l.c. Rt 2.73 min.

### Example 11

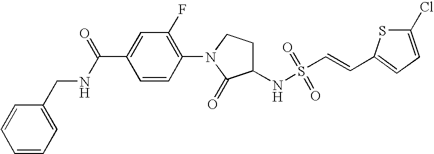

### 4-[3-({[(E)-2-(5-Chloro-2-thienyl)ethenyl]sulfonyl}amino)-2-oxo-1-pyrrolidinyl]-3-fluoro-N-(phenylmethyl)benzamide

Mass spectrum: Found: MH⁺ 534. H.p.l.c. Rt 3.29 min.

### Example 12

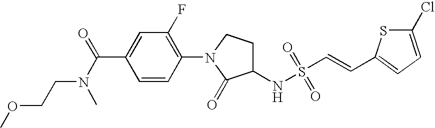

### 4-[3-({[(E)-2-(5-Chloro-2-thienyl)ethenyl]sulfonyl}amino)-2-oxo-1-pyrrolidinyl]-3-fluoro-N-methyl-N-[2-(methyloxy)ethyl]benzamide

Mass spectrum, Found: MH⁺ 516. H.p.l.c. Rt 2.92 min.

### Example 13

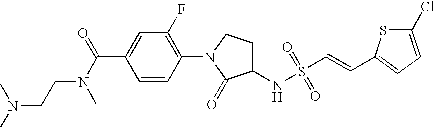

### 4-[3-({[(E)-2-(5-Chloro-2-thienyl)ethenyl]sulfonyl}amino)-2-oxo-1-pyrrolidinyl]-N-[2-(dimethylamino)ethyl]-3-fluoro-N-methylbenzamide

Mass spectrum: Found: MH⁺ 529. H.p.l.c. Rt 2.36 min.

### Example 14

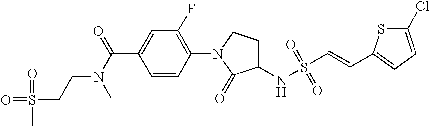

### 4-[3-({[(E)-2-(5-Chloro-2-thienyl)ethenyl]sulfonyl}amino)-2-oxo-1-pyrrolidinyl]-3-fluoro-N-methyl-N-[2-(methylsulfonyl)ethyl]benzamide

Mass spectrum: Found: MH⁺ 564. H.p.l.c. Rt 2.83 min.

### Example 15

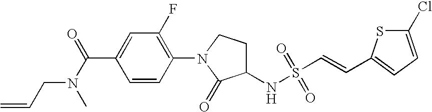

### 4-[3-({[(E)-2-(5-Chloro-2-thienyl)ethenyl]sulfonyl}amino)-2-oxo-1-pyrrolidinyl]-3-fluoro-N-methyl-N-2-propen-1-ylbenzamide

Mass spectrum: Found: MH⁺ 498. H.p.l.c. Rt 3.08 min.

### Example 16

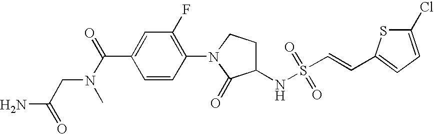

### N-(2-Amino-2-oxoethyl)-4-[3-({[(E)-2-(5-chloro-2-thienyl)ethenyl]sulfonyl}amino)-2-oxo-1-pyrrolidinyl]-3-fluoro-N-methylbenzamide

Mass spectrum: Found: MH⁺ 515 and M+H₂0⁺ 532. H.p.l.c. Rt 2.67 min.

### Example 17

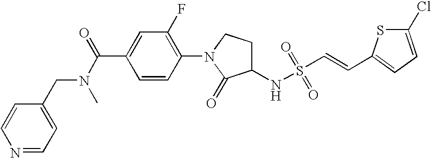

### 4-[3-({[(E)-2-(5-Chloro-2-thienyl)ethenyl]sulfonyl}amino)-2-oxo-1-pyrrolidinyl]-3-fluoro-N-methyl-N-(4-pyridinylmethyl)benzamide

Mass spectrum: Found: MH⁺ 549. H.p.l.c. Rt 2.63 min.

### Example 18

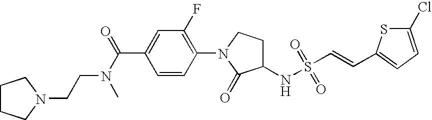

### 4-[3-({[(E)-2-(5-Chloro-2-thienyl)ethenyl]sulfonyl}amino)-2-oxo-1-pyrrolidinyl]-3-fluoro-N-methyl-N-[2-(1-pyrrolidinyl)ethyl]benzamide

Mass spectrum: Found: MH⁺ 555. H.p.l.c. Rt 2.39 min.

### Example 19

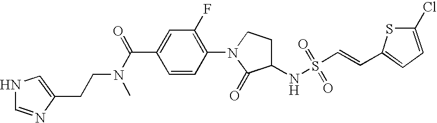

### 4-[3-({[(E)-2-(5-Chloro-2-thienyl)ethenyl]sulfonyl}amino)-2-oxo-1-pyrrolidinyl]-3-fluoro-N-[2-(1H-imidazol-4-yl)ethyl]-N-methylbenzamide

Mass spectrum: Found: MH⁺ 552. H.p.l.c. Rt 2.36 min.

### Example 20

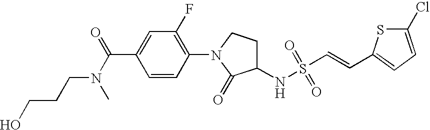

### 4-[3-({[(E)-2-(5-Chloro-2-thienyl)ethenyl]sulfonyl}amino)-2-oxo-1-pyrrolidinyl]-3-fluoro-N-(3-hydroxypropyl)-N-methylbenzamide

Mass spectrum: Found: MH⁺ 516. H.p.l.c. Rt 2.76 min.

### Example 21

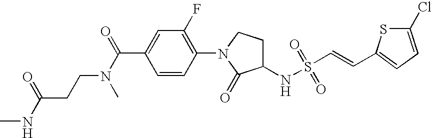

### 4-[3-({[(E)-2-(5-Chloro-2-thienyl)ethenyl]sulfonyl}amino)-2-oxo-1-pyrrolidinyl]-3-fluoro-N-methyl-N-[3-(methylamino)-3-oxopropyl]benzamide

Mass spectrum: Found: MH⁺ 543. H.p.l.c. Rt 2.71 min.

### Example 22

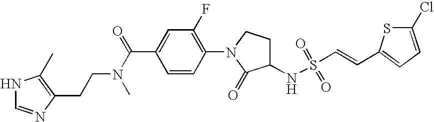

### 4-[3-({[(E)-2-(5-Chloro-2-thienyl)ethenyl]sulfonyl}amino)-2-oxo-1-pyrrolidinyl]-3-fluoro-N-methyl-N-[2-(4-methyl-1H-imidazol-5-yl)ethyl]benzamide

Mass spectrum: Found: MH⁺ 566. H.p.l.c. Rt 2.38 min

### Example 23

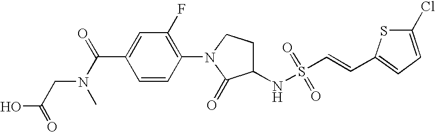

### N-({4-[3-({[(E)-2-(5-Chloro-2-thienyl)ethenyl]sulfonyl}amino)-2-oxo-1-pyrrolidinyl]-3-fluorophenyl}carbonyl)-N-methylglycine

A solution of Intermediate 13 (0.025 g) in DMF (1 ml) was treated with TEA (0.025 ml), a solution of TBTU (0.02 g) in DMF (0.5 ml) and finally with a solution of 1,1-dimethylethyl N-methylglycinate (0.0109 g) in DMF (0.5 ml). The mixture was stirred at ambient for 18 h and then evaporated to dryness. The residue was treated with a mixture of TFA (0.5 ml) and DCM (0.5 ml). After 2 h the mixture was evaporated to dryness and the residue taken up in DMSO (0.5 ml), filtered and the desired product purified from the filtrate by means of mass-directed high performance liquid chromatography to give the title compound (0.0086 g) as a white solid.

Mass spectrum: Found: MH⁺ 516. H.p.l.c. Rt 2.92 min.

Using similar chemistry and Intermediate 13 the following compound was prepared:

### Example 24

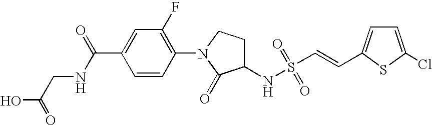

### N-({4-[3-({[(E)-2-(5-Chloro-2-thienyl)ethenyl]sulfonyl}amino)-2-oxo-1-pyrrolidinyl]-3-fluorophenyl}carbonyl)glycine

Mass spectrum: Found: MH⁺ 502. H.p.l.c. Rt 2.95 min.

### Example 25

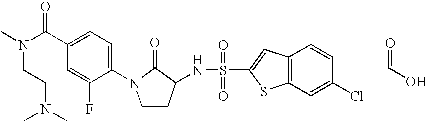

### 4-(3-{[(6-Chloro-1-benzothien-2-yl)sulfonyl]amino}-2-oxo-1-pyrrolidinyl)-N-[2-(dimethylamino)ethyl]-3-fluoro-N-methylbenzamide formate (i.e. Formic acid-4-(3-{[(6-chloro-1-benzothien-2-yl)sulfonyl]amino}-2-oxo-1-pyrrolidinyl)-N-[2-(dimethylamino)ethyl]-3-fluoro-N-methylbenzamide (1:1))

A solution of Intermediate 15 (0.058 g) in dry DMF (0.75 ml) was treated with TBTU (0.077 g) in dry DMF (0.75 ml), N,N,N′-trimethyl-1,2-ethanediamine (0.031 ml) in dry DMF (0.75 ml) and DIPEA (0.043 ml). The reaction was allowed to stir at ambient temperature for 18 h before concentrating under reduced pressure and partitioning the residue between DCM and aqueous saturated sodium bicarbonate solution. The separated organic layer was passed through a hydrophobic frit, concentrated under reduced pressure and the residue was purified by means of mass-directed high performance liquid chromatography to give the title compound (0.041 g) as a white solid.

Mass spectrum: Found: MH⁺ 553. H.p.l.c. Rt 2.48 min.

Using similar chemistry and Intermediate 15 the following compounds were prepared:

### Example 26

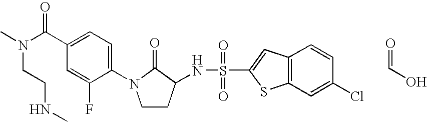

### 4-(3-{[(6-Chloro-1-benzothien-2-yl)sulfonyl]amino}-2-oxo-1-pyrrolidinyl)-3-fluoro-N-methyl-N-[2-(methylamino)ethyl]benzamide formate

Mass spectrum: Found: MH⁺ 539. H.p.l.c. Rt 2.46 min.

### Example 27

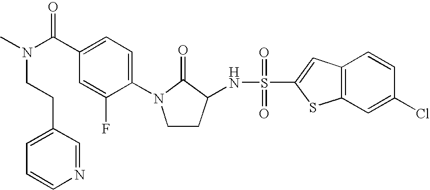

### 4-(3-{[(6-Chloro-1-benzothien-2-yl)sulfonyl]amino}-2-oxo-1-pyrrolidinyl)-3-fluoro-N-methyl-N-[2-(3-pyridinyl)ethyl]benzamide

Mass spectrum: Found: MH⁺ 587. H.p.l.c. Rt 2.80 min.

### Example 28

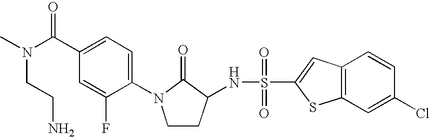

### N-(2-Aminoethyl)-(4-(3-{[(6-chloro-1-benzothien-2-yl)sulfonyl]amino}-2-oxo-1-pyrrolidinyl)-3-fluoro-N-methylbenzamide

A solution of Intermediate 15 (0.058 g) in dry DMF (0.75 ml) was treated with TBTU (0.077 g) in dry DMF (0.75 ml), 1,1-dimethylethyl[2-(methylamino)ethyl]carbamate (0.043 g) in dry DMF (0.75 ml) and DIPEA (0.043 ml). The reaction was allowed to stir at ambient temperature for 18 h before concentrating under reduced pressure and partitioning the residue between DCM and aqueous saturated sodium bicarbonate solution. The separated organic layer was passed through a hydrophobic frit and concentrated under reduced pressure. The residue was treated with 1:1 TFA:DCM (4 ml) stirring for 2 h, re-concentrated under reduced pressure and purified by means of mass-directed high performance liquid chromatography to give the title compound (0.042 g) as a white solid.

Mass spectrum: Found: MH⁺ 525. H.p.l.c. Rt 2.46 min.

### Example 29

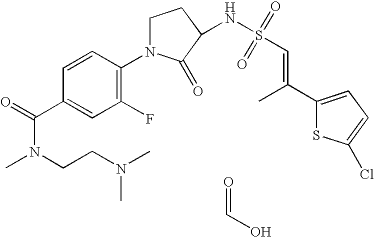

### 4-[3-({[(1E)-2-(5-chloro-2-thienyl)-1-propen-1-yl]sulfonyl}amino)-2-oxo-1-pyrrolidinyl]-N-[2-(dimethylamino)ethyl]-3-fluoro-N-methylbenzamide formate

A solution of Intermediate 17 (0.092 g) in 1:1 TFA/DCM (4 ml) was allowed to stir for 18 h at ambient temperature. The solvent was removed by blowing down using nitrogen gas to offer a gummy solid. This material was basified using SCX SPE (5 g/20 cc) eluting the product with 10% ammonia/MeOH and concentrating under reduced pressure to provide a colourless gum (0.041 g). This material was dissolved in dry MeCN (3 ml) and treated with pyridine (0.024 ml) and Intermediate 9 (0.032 g) allowing to stir at ambient temperature for 18 hr. The reaction was concentrated under reduced pressure and purified by means of mass-directed high performance liquid chromatography to give thee title compound (0.048 g) as a white solid.

Mass spectrum: Found: MH⁺ 543. H.p.l.c. Rt 2.40 min.

Using similar chemistry the following compounds were prepared:

### Example 30

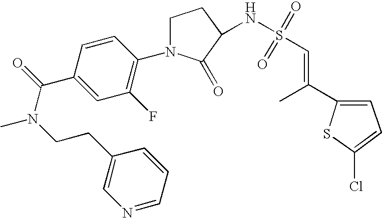

### 4-[3-({[(1E)-2-(5-Chloro-2-thienyl)-1-propen-1-yl]sulfonyl}amino)-2-oxo-1-pyrrolidinyl]-3-fluoro-N-methyl-N-[2-(3-pyridinyl)ethyl]benzamide

From Intermediate 18

Mass spectrum: Found: MH⁺ 577. H.p.l.c. Rt 2.71 min.

### Example 31

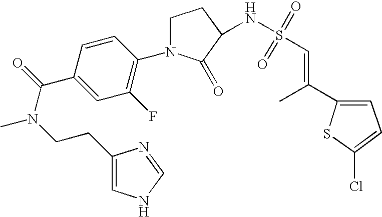

### 4-[3-({[(1E)-2-(5-Chloro-2-thienyl)-1-propen-1-yl]sulfonyl}amino)-2-oxo-1-pyrrolidinyl]-3-fluoro-N-[2-(1H-imidazol-4-yl)ethyl]-N-methylbenzamide

From Intermediate 19

Mass spectrum: Found: MH⁺ 566. H.p.l.c. Rt 2.42 min.

### Example 32

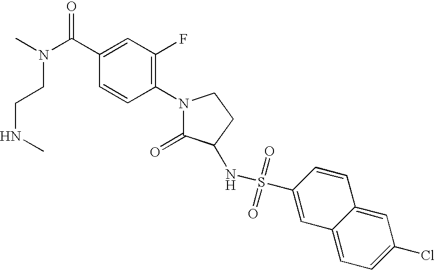

### 4-(3-{[(6-Chloro-2-naphthalenyl)sulfonyl]amino}-2-oxo-1-pyrrolidinyl)-3-fluoro-N-methyl-N-[2-(methylamino)ethyl]benzamide

Prepared in a two step process, initially according to the procedure for Example 25, from Intermediate 23 and N,N′-dimethyl-N-(phenylmethyl)-1,2-ethanediamine dihydrochloride (with 4 equivalents of DIPEA). The resulting amide was taken up in ethanol (5 ml) and treated with 10% Pd/C (50 wt % H₂O). The mixture was hydrogenated at atmospheric temperature and pressure for 2 hours. The mixture was then filtered, and solvent evaporated under reduced pressure. Purification via Mass Directed high performance liquid chromatography gave the title compound.

Mass spectrum: Found: MH⁺ 533. H.p.l.c. Rt 2.52 min.

In Vitro Assay for Inhibition of Factor Xa

Compounds of the present invention were tested for their Factor Xa inhibitory activity as determined in vitro by their ability to inhibit human Factor Xa in a fluorogenic assay, using Rhodamine 110, bis-CBZ-glycylglycyl-L-arginine amide as the fluorogenic substrate. Compounds were diluted from a 10 mM stock solution in dimethylsulfoxide at appropriate concentrations. Assay was performed at room temperature using buffer consisting of of: 50 mM Tris-HCl, 150 mM NaCl, 5 mM CaCl₂, pH 7.4. containing human Factor Xa (final conc. Of 0.0003 U·ml-1). Compound and enzyme were preincubated for 15 min prior to addition of the substrate (final conc. of 10 μM). The reaction was stopped after 3 hrs with the addition of H-D-Phe-Pro-Arg-Chloromethylketone. An LJL-Analyst fluorimeter was used to monitor fluorescence with 485 nm excitation/535 nm emission. To obtain IC₅₀ values the data were analysed using ActivityBase® and XLfit®.

Calculation of Ki values:
Ki=IC₅₀/(1+[Substrate]/Km)

The Ki value for the above assay can be obtained by dividing the IC₅₀ value by 1.6.

All of the synthetic Example compounds 1-31 were tested by the above described in vitro assay and were found to exhibit Factor Xa inhibitory activity. Preferably compounds have a Ki value of less than 1 μM (Examples 1, 2, 3, 4, 5, 6, 7, 8, 9, 10, 12, 13, 14, 15, 16, 17, 15 18, 19, 20, 21, 22, 23, 25, 26, 27, 28, 29, 30, 31). More preferably, compounds have a Ki value of less than 0.1 μM (Examples 1, 3, 4, 6, 15, 26, 27, 29, 30, 31).

Method for Measurement of Prothrombin Time (PT)

Prothrombin time of compounds according to the invention may be determined using the following assay.

Blood is collected into a sodium citrate solution (ratio 9:1) to give a final concentration of 0.38% citrate. Plasma is generated by centrifugation of citrated blood samples at 1200×g for 20 min at 4° C. and stored at −20° C. until use. PT analysis is conducted using plasma pooled from 4 separate donors (2 male and 2 female).

The PT test is performed using the BCS Coagulation Analyzer (Dade Behring). For assay, 50 ul of plasma containing test compound at concentrations ranging from 0.03 to 100 uM (made from a 100 uM stock containing 1% DMSO in plasma) is combined with 100 ul of Thromboplastin C Plus (Dade Behring). Upon addition of the reagents, absorbance at 405 nm is monitored and time to clot formation is determined (normal range for human plasma is 10.6-12.4 seconds).

General Purification and Analytical Methods

LC/MS Method.

Analytical HPLC was conducted on a Supelcosil LCABZ+PLUS column (3 μm, 3.3 cm×4.6 mm ID) eluting with 0.1% HCO₂H and 0.01 M ammonium acetate in water (solvent A), and 95% MeCN and 0.05% HCO₂H in water (solvent B), using the following elution gradient 0-0.7 minutes 0% B, 0.7-4.2 minutes 0→100% B, 4.2-5.3 minutes 100% B, 5.3-5.5 minutes 100→0% B at a flow rate of 3 ml/minutes (System 1). The mass spectra (MS) were recorded on a Fisons VG Platform mass spectrometer using electrospray positive ionisation [(ES+ve to give MH⁺ and M(NH₄)⁺ molecular ions] or electrospray negative ionisation [(ES−ve to give (M−H⁻ molecular ion] modes.

¹H nmr spectra were recorded using a Bruker DPX 400 MHz spectrometer using tetramethylsilane as the external standard.

Biotage™ chromatography refers to purification carried out using equipment sold by Dyax Corporation (either the Flash 40i or Flash 150i) and cartridges pre-packed with KPSil.

Mass directed high performance liquid chromatography was conducted on a HPLCABZ+5 μm column (5 cm×10 mm i.d.) with 0.1% HCO₂H in water and 95% MeCN, 5% water (0.5% HCO₂H) utilising the following gradient elution conditions: 0-1.0 minutes 5% B, 1.0-8.0 minutes 5→30% B, 8.0-8.9 minutes 30% B, 8.9-9.0 minutes 30→95% B, 9.0-9.9 minutes 95% B, 9.9-10 minutes 95→0% B at a flow rate of 8 ml minutes⁻¹ (System 2). The Gilson 202-fraction collector was triggered by a VG Platform Mass Spectrometer on detecting the mass of interest.

SPE (solid phase extraction) refers to the use of cartidges sold by International Sorbent Technology Ltd.

SCX SPE (solid phase extraction) refers to the use of acidic ion exchange cartridges sold by International Sorbent Technology Ltd.

1. A compound of formula (1): 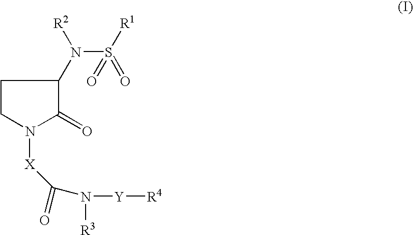
wherein:
R¹ represents a group selected from: 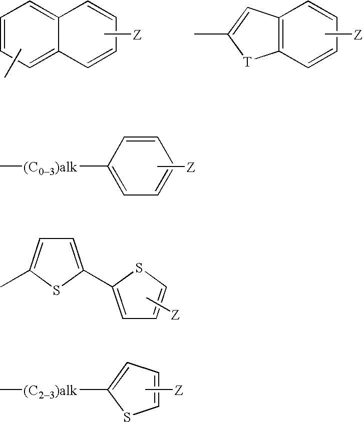
each ring of which optionally contains a further heteroatom N,
Z represents an optional substituent halogen,
alk represents alkylene or alkenylene,
T represents S, O or NH;
R² represents hydrogen, —C₁₋₆alkyl, —C₁₋₃alkylCONR^{a}R^{b}, —C₁₋₃alkylCO₂C₁₋₄alkyl, —C₂₋₃alkylmorpholino, —CO₂C₁₋₄alkyl, or —C₁₋₃alkylCO₂H;
R^{a} and R^{b} independently represent hydrogen, —C₁₋₆alkyl, or together with the N atom to which they are bonded form a 5-, 6- or 7-membered non-aromatic heterocyclic ring optionally containing an additional heteroatom selected from O, N or S, optionally substituted by —C₁₋₄alkyl, and optionally the S heteroatom is substituted by O, i.e. represents S(O)ₙ;
n represents 0-2;
X represents phenyl or a 5- or 6-membered aromatic heterocyclic group containing at least one heteroatom selected from O, N or S, each of which is optionally substituted by 0-2 groups selected from: halogen, —C₁₋₄alkyl, —C₂₋₄alkenyl, —CN, —CF₃, —NR^{a}R^{b}, —C₀₋₄alkylOR^{e}, —C(O)R^{f} and —C(O)NR^{a}R^{b};
R^{e} represents hydrogen or —C₁₋₆alkyl;
R^{f} represents —C₁₋₆alkyl;
Y is absent or represents —C₁₋₃alkylene-;
R³ represents hydrogen or —C₁₋₆alkyl;
R⁴ represents —C₃₋₄alkenyl, —CH₂CH₂OH, —CH₂CO₂H, —CH₂CH₂OC₁₋₃alkyl, —CH₂CH₂SO₂C₁₋₃alkyl, —CH₂CH₂NR^{c}R^{d}, —CH₂CONR^{c}R^{d}, phenyl or a 5- or 6-membered aromatic or non-aromatic heterocyclic group containing at least one heteroatom selected from O, N or S and optionally substituted by —C₁₋₄alkyl;
R^{c} and R^{d} independently represent hydrogen, —C₁₋₆alkyl, or together with the N atom to which they are bonded form a 5-, 6- or 7-membered non-aromatic heterocyclic ring optionally containing an additional heteroatom selected from O, N or S, optionally substituted by —C₁₋₄alkyl;
and/or pharmaceutically acceptable derivative thereof.

2. A compound according to claim 1 wherein R¹ represents a group selected from: 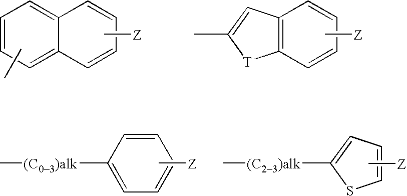
each ring of which optionally contains a further heteroatom N,
Z represents an optional substituent halogen,
alk represents alkylene or alkenylene,
T represents S, O or NH;
and/or pharmaceutically acceptable derivative thereof.

3. A compound according to claim 1 or claim 2 wherein R² represents hydrogen and/or pharmaceutically acceptable derivative thereof.

4. A compound according to any one of claims 1-3 wherein X represents phenyl or a 5- or 6-membered aromatic heterocyclic group containing at least one heteroatom selected from O, N or S, each of which is optionally substituted by 0-2 groups selected from: halogen, —C₁₋₄alkyl or —NR^{a}R^{b} and/or pharmaceutically acceptable derivative thereof.

5. A compound according to any one of claims 1-4 wherein Y is absent or represents C₁₋₂ alkylene and/or pharmaceutically acceptable derivative thereof.

6. A compound according to any one of claims 1-5 wherein R³ represents hydrogen or methyl and/or pharmaceutically acceptable derivative thereof.

7. A compound according to any one of claims 1-6 wherein R⁴ represents —C₃₋₄alkenyl, —CH₂CH₂OH, —CH₂CO₂H, —CH₂CH₂OCH₃, —CH₂CH₂SO₂CH₃, —CH₂CH₂NR^{c}R^{d}, —CH₂CONR^{c}R^{d}, phenyl or a 5- or 6-membered aromatic heterocyclic group containing one or two heteroatoms selected from O, N or S and optionally substituted by —C₁₋₄alkyl and/or pharmaceutically acceptable derivative thereof.

8. A compound according to claim 1 selected from:
4-[3-({[(1E)-2-(5-Chloro-2-thienyl)-1-propen-1-yl]sulfonyl}amino)-2-oxo-1-pyrrolidinyl]-3-fluoro-N-methyl-N-[2-(methylamino)ethyl]benzamide;
4-[3-({[(1E)-2-(5-Chloro-2-thienyl)-1-propen-1-yl]sulfonyl}amino)-2-oxo-1-pyrrolidinyl]-3-fluoro-N-(2-hydroxyethyl)-N-methylbenzamide;
4-[3-({[(1E)-2-(5-Chloro-2-thienyl)-1-propen-1-yl]sulfonyl}amino)-2-oxo-1-pyrrolidinyl]-3-fluoro-N-methyl-N-(2-pyridinylmethyl)benzamide;
4-[3-({[(1E)-2-(5-Chloro-2-thienyl)-1-propen-1-yl]sulfonyl}amino)-2-oxo-1-pyrrolidinyl]-3-fluoro-N-methyl-N-[2-(methylsulfonyl)ethyl]benzamide;
4-[3-({[(1E)-2-(5-Chloro-2-thienyl)-1-propen-1-yl]sulfonyl}amino)-2-oxo-1-pyrrolidinyl]-3-fluoro-N-methyl-N-[2-(methyloxy)ethyl]benzamide;
4-[3-({[(E)-2-(5-Chloro-2-thienyl)ethenyl]sulfonyl}amino)-2-oxo-1-pyrrolidinyl]-3-fluoro-N-methyl-N-[2-(3-pyridinyl)ethyl]benzamide;
4-[3-({[(E)-2-(5-Chloro-2-thienyl)ethenyl]sulfonyl}amino)-2-oxo-1-pyrrolidinyl]-3-fluoro-N-methyl-N-(2-phenylethyl)benzamide;
4-[3-({[(E)-2-(5-Chloro-2-2-thienyl)ethenyl]sulfonyl}amino)-2-oxo-1-pyrrolidinyl]-3-fluoro-N-(4-pyridinylmethyl)benzamide;
4-[3-({[(E)-2-(5-Chloro-2-thienyl)ethenyl]sulfonyl}amino)-2-oxo-1-pyrrolidinyl]-3-fluoro-N-(3-pyridinylmethyl)benzamide;
4-[3-({[(E)-2-(5-Chloro-2-thienyl)ethenyl]sulfonyl}amino)-2-oxo-1-pyrrolidinyl]-3-fluoro-N-(2-hydroxyethyl)-N-methylbenzamide;
4-[3-({[(E)-2-(5-Chloro-2-thienyl)ethenyl]sulfonyl}amino)-2-oxo-1-pyrrolidinyl]-3-fluoro-N-(phenylmethyl)benzamide;
4-[3-({[(E)-2-(5-Chloro-2-thienyl)ethenyl]sulfonyl}amino)-2-oxo-1-pyrrolidinyl]-3-fluoro-N-methyl-N-[2-(methyloxy)ethyl]benzamide;
4-[3-({[(E)-2-(5-Chloro-2-thienyl)ethenyl]sulfonyl}amino)-2-oxo-1-pyrrolidinyl]-N-[2-(dimethylamino)ethyl]-3-fluoro-N-methylbenzamide;
4-[3-({[(E)-2-(5-Chloro-2-thienyl)ethenyl]sulfonyl}amino)-2-oxo-1-pyrrolidinyl]-3-fluoro-N-methyl-N-[2-(methylsulfonyl)ethyl]benzamide;
4-[3-({[(E)-2-(5-Chloro-2-thienyl)ethenyl]sulfonyl}amino)-2-oxo-1-pyrrolidinyl]-3-fluoro-N-methyl-N-2-propen-1-ylbenzamide;
N-(2-Amino-2-oxoethyl)-4-[3-({[(E)-2-(5-chloro-2-thienyl)ethenyl]sulfonyl}amino)-2-oxo-1-pyrrolidinyl]-3-fluoro-N-methylbenzamide;
4-[3-({[(E)-2-(5-Chloro-2-thienyl)ethenyl]sulfonyl}amino)-2-oxo-1-pyrrolidinyl]-3-fluoro-N-methyl-N-(4-pyridinylmethyl)benzamide;
4-[3-({[(E)-2-(5-Chloro-2-thienyl)ethenyl]sulfonyl}amino)-2-oxo-1-pyrrolidinyl]-3-fluoro-N-methyl-N-[2-(1-pyrrolidinyl)ethyl]benzamide;
4-[3-({[(E)-2-(5-Chloro-2-thienyl)ethenyl]sulfonyl}amino)-2-oxo-1-pyrrolidinyl]-3-fluoro-N-[2-(1H-imidazol-4-yl)ethyl]-N-methylbenzamide;
4-[3-({[(E)-2-(5-Chloro-2-thienyl)ethenyl]sulfonyl}amino)-2-oxo-1-pyrrolidinyl]-3-fluoro-N-(3-hydroxypropyl)-N-methylbenzamide;
4-[3-({[(E)-2-(5-Chloro-2-thienyl)ethenyl]sulfonyl}amino)-2-oxo-1-pyrrolidinyl]-3-fluoro-N-methyl-N-[3-(methylamino)-3-oxopropyl]benzamide;
4-[3-({[(E)2-(5-Chloro-2-thienyl)ethenyl]sulfonyl}amino)-2-oxo-1-pyrrolidinyl]-3-fluoro-N-methyl-N-[2-(4-methyl-1H-imidazol-5-yl)ethyl]benzamide;
5N-({4-[3-({[(E)-2-(5-Chloro-2-thienyl)ethenyl]sulfonyl}amino)-2-oxo-1-pyrrolidinyl]-3-fluorophenyl}carbonyl)-N-methylglycine;
N-({4-[3-({[(E)-2-(5-Chloro-2-thienyl)ethenyl]sulfonyl}amino)-2-oxo-1-pyrrolidinyl]-3-fluorophenyl}carbonyl)glycine;.
4-(3-{[(6-Chloro-1-benzothien-2-yl)sulfonyl]amino}-2-oxo-1-pyrrolidinyl)-N-[2-(dimethylamino)ethyl]-3-fluoro-N-methylbenzamide;
4-(3-{[(6-Chloro-1-benzothien-2-yl)sulfonyl]amino}-2-oxo-1-pyrrolidinyl)-3-fluoro-N-methyl-N-[2-(methylamino)ethyl]benzamide;
4-(3-{[(6-Chloro-1-benzothien-2-yl)sulfonyl]amino}-2-oxo-1-pyrrolidinyl)-3-fluoro-N-methyl-N-[2-(3-pyridinyl)ethyl]benzamide;
N-(2-Aminoethyl)-4-(3-{[(6-chloro-1-benzothien-2-yl)sulfonyl]amino}-2-oxo-1-pyrrolidinyl)-3-fluoro-N-methylbenzamide;
4-[3-({[(1E)-2-(5-Chloro-2-thienyl)-1-propen-1-yl]sulfonyl}amino)-2-oxo-1-pyrrolidinyl]-N-[2-(dimethylamino)ethyl]-3-fluoro-N-methylbenzamide;
4-[3-({[(1E)-2-(5-Chloro-2-thienyl)-1-propen-1-yl]sulfonyl}amino)-2-oxo-1-pyrrolidinyl]-3-fluoro-N-methyl-N-[2-(3-pyridinyl)ethyl]benzamide;
4-[3-({[(1E)-2-(5-Chloro-2-thienyl)-1-propen-1-yl]sulfonyl}amino)-2-oxo-1-pyrrolidinyl]-3-fluoro-N-[2-(1H-imidazol-4-yl)ethyl]-N-methylbenzamide;
4-(3-{[(6-Chloro-2-naphthalenyl)sulfonyl]amino}-2-oxo-1-pyrrolidinyl)-3-fluoro-N-methyl-N-[2-(methylamino)ethyl]benzamide;
and/or pharmaceutically acceptable derivative thereof.

9. A compound according to any one of claims 1-8 and/or pharmaceutically acceptable derivative thereof for use in therapy.

10. A pharmaceutical composition comprising a compound according to any one of claims 1-8 and/or pharmaceutically acceptable derivative thereof together with a pharmaceutical carrier and/or excipient.

11. Use of a compound according to any one of claims 1-8 and/or pharmaceutically acceptable derivative thereof for the manufacture of a medicament for the treatment of a patient suffering from a condition susceptible to amelioration by a Factor Xa inhibitor.

12. A method of treating a patient suffering from a condition susceptible to amelioration by a Factor Xa inhibitor comprising administering a therapeutically effective amount of a compound according to any one of claims 1-8 and/or pharmaceutically acceptable derivative thereof.

13. A process for preparing a compound of formula (I) which comprises:
(a) reacting compound of formula (II) or an acid addition salt thereof with a compound of formula (III) where V is a suitable leaving group: 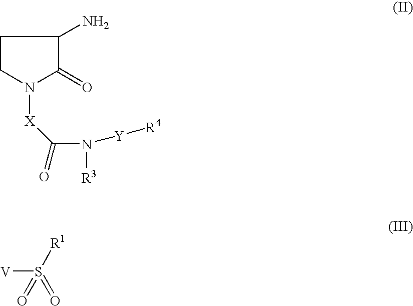
OR:
(b) by reacting compounds of formula (XIII) with compounds of formula (VI): 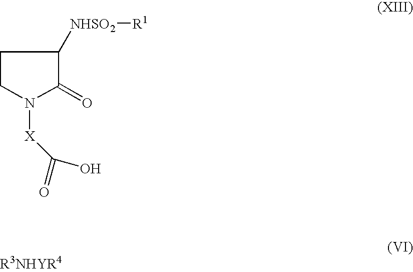
(c) by reacting a compound of formula (I) where R² is hydrogen with a compound of formula (XVII):
R²-T (XVII)
where R² is —C₁₋₆alkyl, —C₁₋₃alkylCONR^{a}R^{b}, —C₁₋₃alkylCO₂C₁₋₄alkyl, —C₂₋₃alkylmorpholino or —CO₂C₁₋₄alkyl and T is a suitable leaving group, optionally followed by removal of the alkyl protecting group where appropriate.
